SEQUENCE LISTING (72 sequences)

SEQ ID NO: 1 - protein, 310 aa, Homo sapiens [sequence omitted]

SEQ ID NO: 2 - protein, 27 aa, Artificial Sequence
  amino acid residues from 284 to 310 of SEQ ID NO 1
GISHVEAKKRILNLHPKIFGNKWFPRV

SEQ ID NO: 3 - protein, 251 aa, Artificial Sequence
  ScFv1
SYELTQPPSVSVAPGKTARITCGGNNIGSKSVHWYQQKPGQAPVLVVYDDSDRPSGIPERFSGSNSGNTATLTISRVEAGDEADYYCQVWDSSSDHVVFGGGTKLTVLGSGGSTITSYNVYYTKLSSSGTQVQLQESGGGLVQPGRSLRLSCAASGFTFDDYAMHWVRQAPGKGLEWVSGISWNSGSIGYADSVKGRFTISRDNAKNSLYLQMNSLRAEDTALYYCAKVSAYSSSFDYWGQGTLVTVSSAS

SEQ ID NO: 4 - DNA, 753 bp, Artificial Sequence
  scFv1
tcctatgagctgactcagccaccctcggtgtcagtggccccaggaaagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatgtggtattcggcggagggaccaagctcaccgtcctaggttccggagggtcgaccataacttcgtataatgtatactatacgaagttatcctcgagcggtacccaggtgcagctgcaggagtctgggggaggcttggtacagcctggcaggtccctgagactttcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagtatccgcgtatagcagctcgtttgactactggggccagggaaccctggtcaccgtctcctcagctagc

SEQ ID NO: 5 - protein, 257 aa, Artificial Sequence
  scFv2
QSALTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLGSGGSTITSYNVYYTKLSSSGTQVQLLESGAEVKKPGASVKVSCKASGYTFTAYYMHWVRQAPGQGLEWMAMIDPSGSITSYAQKFQGRVTMSRDTSTSTLYMELSSLRSDDTAVYYCARDLKEGFSVPGAFDIWGQGTMVTVSSAS

SEQ ID NO: 6 - DNA, 771 bp, Artificial Sequence
  scFv2
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaagcggccctcaggggtttctaatcgcttctctggctccaagtctggcaacacggcctccctgaccatctctgggctccaggctgaggacgaggctgattattactgcagctcatatacaagcagcagcactttggtgttcggcggagggaccaagctgaccgtcctaggttccggagggtcgaccataacttcgtataatgtatactatacgaagttatcctcgagcggtacccaggtgcagctgttggagtctggggctgaggtgaagaagcccggggcctcagtgaaggtttcctgcaaggcatctggatacaccttcaccgcctactatatgcactgggtgcgacaggcccctggacaagggcttgagtggatggcaatgatcgaccccagtggtagtatcacaagctacgcacagaagttccagggcagagtcaccatgagcagggacacgtccacgagcacactctacatggagctgagcagcctgagatctgacgacacggccgtgtattactgtgcgagagatctgaaagaggggtttagtgtccctggggcttttgatatctggggccaagggacaatggtcactgtctcttcagctagc

SEQ ID NO: 7 - protein, 257 aa, Artificial Sequence
  scFv3
QSVLTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLGSGGSTITSYNVYYTKLSSSGTEVQLVQSGGGVAQPGRSLRLSCAASGITFNSYGMHWVRQAPGKGLEWVAVMPYDGSNEYYADSVKGRFTISRDNSKNTLYLQMNSLRAEDTAVYYCAKGSGMVQLWADAFDVWGQGTMVTVSSAS

SEQ ID NO: 8 - DNA, 771 bp, Artificial Sequence
  scFv3
cagtctgtgttgacgcaccctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaagcggccctcaggggtttctaatcgcttctctggctccaagtctggcaacacggcctccctgaccatctctgggctccaggctgaggacgaggctgattattactgcagctcatatacaagcagcagcactctggtattcggcggagggaccaagctcaccgtcctaggttccggagggtcgaccataacttcgtataatgtatactatacgaagttatcctcgagcggtaccgaggtccagctggtacagtctgggggaggcgtggcccagcctgggaggtccctgagactctcctgtgcagcctctggaatcaccttcaatagctatggcatgcactgggtccgccaggctccaggcaaggggctggagtgggtggcagttatgccatatgatggaagtaatgaatactatgcagactccgtgaagggccgattcaccatctccagagacaattccaagaacacactgtatctgcaaatgaacagcctgagagctgaggacacggctgtgtattactgtgcgaaaggttccgggatggttcagctatgggcggatgcttttgatgtctggggccaagggacaatggtcaccgtctcttcagctagc

SEQ ID NO: 9 - protein, 251 aa, Artificial Sequence
  scFv4
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVFGGGTKLTVLGSGGSTITSYNVYYTKLSSSGTQVQLVQSGAEVKKPGSSVKVSCKASGGTFSSYATSWVRQAPGQGLEWMGGIIPTFGTANYAQKFQGRVTITADESTSTAYMELSSLRSEDTAVYYCAREGLGYFDYWGQGTLVTVSSAS

SEQ ID NO: 10 - DNA, 753 bp, Artificial Sequence
  scFv4
cagtctgtgttgacgcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtccggtgttcggcggagggaccaagctgaccgtcctaggttccggagggtcgaccataacttcgtataatgtatactatacgaagttatcctcgagcggtacccaggtccagctggtacagtctggggctgaggtgaagaagcctgggtcctcggtgaaggtctcctgcaaggcttctggaggcaccttcagcagctatgctaccagctgggtgcgacaggcccctggacaagggcttgagtggatgggagggatcatccctacctttggtacagcaaactacgcacagaagttccagggcagagtcacgattaccgcggacgaatccacgagcacagcctacatggagctgagcagcctgagatctgaggacacggccgtgtattactgtgcgagagagggtctgggctactttgactactggggccagggaaccctggtcaccgtctcctcagctagc

SEQ ID NO: 11 - protein, 252 aa, Artificial Sequence
  scFv5
QSALTQPPSASGTPGQRVTVSCSGSSSNIGSNTVNWYQQLPGTAPKLLIYSNNQRPSGVPDRFSASKSGTSASLAISGLQSEDEADYYCAAWDDSLNGHVVFGGGTKLTVLGSGGSTITSYNVYYTKLSSSGTQVQLVQSGAEVKKPGESLKISCKGSGYSFTSYWIGWVRQMPGKGLEWMGIIYPGDSDTRYSPSFQGQVTISADKSISTAYLQWSSLKASDTAMYYCASGIYDAFDIWGQGTMVTVSSAS

SEQ ID NO: 12 - DNA, 756 bp, Artificial Sequence
  scFv5
cagtctgccctgactcagccaccctcagcgtctgggacccccgggtagagggtcaccgtctcttgttctggaagcagctccaacatcggaagtaatactgtaaactggtaccagcagctcccaggaacggcccccaaactcctcatctatagtaataatcagcggccctcaggggtccctgaccgattctctgcctccaagtctggcacctcagcctccctggccatcagtgggctccagtctgaggatgaggctgattattactgtgcagcatgggatgatagcctgaatggtcatgtggtattcggcggagggaccaagctcaccgtcctaggttccggagggtcgaccataacttcgtataatgtatactatacgaagttatcctcgagcggtacccaggtgcagctggtacagtctggggcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagcggaatctatgatgcttttgatatctggggccaagggacaatggtcaccgtctcctcagctagc

SEQ ID NO: 13 - protein, 249 aa, Artificial Sequence
  scFv6
EIVMTQSPGTLSLSPGERATLSCRASQSVDSNFLAWYQQRPGQAPRLLIYGVSSSATGIPDRFSGSGSGTDFTLSIDRLEPEDFAVYYCQQYGSSPTFGGGTKLEIKRSGGSTITSYNVYYTKLSSSGTQVQLQESGGGLVQPGRSLRLSCAASGFTFDDYAMHWVRQAPGKGLEWVSGISWNSGSIGYADSVKGRFTISRDNAKNSLYLQMNSLRAEDTAVYYCARSGDYGAFDIWGQGTMVTVSSAS

SEQ ID NO: 14 - DNA, 747 bp, Artificial Sequence
  scFv6
gaaattgtgatgacgcagtctcccggcaccctgtctttgtctccaggggagagagccaccctctcctgcagggccagtcagagtgttgacagcaacttcttagcctggtatcagcaaagacctggccaggctccccggctcctcatctatggtgtatccagcagcgccactggcatcccagacaggttcagtggcagtgggtctgggacagacttcactctctccatcgacagactggagcctgaagattttgctgtgtattactgtcagcaatatggtagctcacccactttcggcggagggaccaagctggaaatcaaacgttccggagggtcgaccataacttcgtataatgtatactatacgaagttatcctcgagcggtacccaggtgcagctgcaggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactcactgtatctgcaaatgaacagcctgagagccgaggacacggctgtttattactgtgcgagatccggtgactacggggcttttgatatctggggccaagggacaatggtcaccgtctcctcagctagc

SEQ ID NO: 15 - protein, 254 aa, Artificial Sequence
  scFv7
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTKLTVLGSGGSTITSYNVYYTKLSSSGTEVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKEGDSSGWSPDYWGQGTLVTVSSAS

SEQ ID NO: 16 - DNA, 762 bp, Artificial Sequence
  scFv7
cagtctgtgctgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctggggtgttcggcggagggaccaagctgaccgtcctaggttccggagggtcgaccataacttcgtataatgtatactatacgaagttatcctcgagcggtaccgaggtgcagctggtacagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagagggagatagcagtggctggtcccctgactactggggccagggaaccctggtcaccgtctcttcagctagc

SEQ ID NO: 17 - protein, 11 aa, Artificial Sequence
  scFv1 LCDRA1
GGNNIGSKSVH

SEQ ID NO: 18 - protein, 7 aa, Artificial Sequence
  scFv1 LCDRA2
DDSDRPS

SEQ ID NO: 19 - protein, 11 aa, Artificial Sequence
  scFv1 LCDRA3
QVWDSSSDHVV

SEQ ID NO: 20 - protein, 5 aa, Artificial Sequence
  scFv1 HCDRA1
DYAMH

SEQ ID NO: 21 - protein, 17 aa, Artificial Sequence
  scFv1 HCDRA2
GISWNSGSIGYADSVKG

SEQ ID NO: 22 - protein, 10 aa, Artificial Sequence
  scFv1 HCDRA3
VSAYSSSFDY

SEQ ID NO: 23 - protein, 14 aa, Artificial Sequence
  scFv2 LCDRA1
TGTSSDVGGYNYVS

SEQ ID NO: 24 - protein, 7 aa, Artificial Sequence
  scFv2 LCDRA2
DVSKRPS

SEQ ID NO: 25 - protein, 10 aa, Artificial Sequence
  scFv2 LCDRA3
SSYTSSSTLV

SEQ ID NO: 26 - protein, 5 aa, Artificial Sequence
  scFv2 HCDRA1
AYYMH

SEQ ID NO: 27 - protein, 17 aa, Artificial Sequence
  scFv2 HCDRA2
MIDPSGSITSYAQKFQG

SEQ ID NO: 28 - protein, 14 aa, Artificial Sequence
  scFv2 HCDRA3
DLKEGFSVPGAFDI

SEQ ID NO: 29 - protein, 14 aa, Artificial Sequence
  scFv3 LCDRA1
TGTSSDVGGYNYVS

SEQ ID NO: 30 - protein, 7 aa, Artificial Sequence
  scFv3 LCDRA2
DVSKRPS

SEQ ID NO: 31 - protein, 10 aa, Artificial Sequence
  scFv3 LCDRA3
SSYTSSSTLV

SEQ ID NO: 32 - protein, 5 aa, Artificial Sequence
  scFv3 HCDRA1
SYGMH

SEQ ID NO: 33 - protein, 17 aa, Artificial Sequence
  scFv3 HCDRA2
VMPYDGSNEYYADSVKG

SEQ ID NO: 34 - protein, 14 aa, Artificial Sequence
  scFv3 HCDRA3
GSGMVQLWADAFDV

SEQ ID NO: 35 - protein, 13 aa, Artificial Sequence
  scFv4 LCDRA1
SGSSSNIGSNTVN

SEQ ID NO: 36 - protein, 7 aa, Artificial Sequence
  scFv4 LCDRA2
SNNQRPS

SEQ ID NO: 37 - protein, 11 aa, Artificial Sequence
  scFv4 LCDRA3
AAWDDSLNGPV

SEQ ID NO: 38 - protein, 5 aa, Artificial Sequence
  scFv4 HCDRA1
SYATS

SEQ ID NO: 39 - protein, 17 aa, Artificial Sequence
  scFv4 HCDRA2
GIIPTFGTANYAQKFQG

SEQ ID NO: 40 - protein, 8 aa, Artificial Sequence
  scFv4 HCDRA3
EGLGYFDY

SEQ ID NO: 41 - protein, 13 aa, Artificial Sequence
  scFv5 LCDRA1
SGSSSNIGSNTVN

SEQ ID NO: 42 - protein, 7 aa, Artificial Sequence
  scFv5 LCDRA2
SNNQRPS

SEQ ID NO: 43 - protein, 12 aa, Artificial Sequence
  scFv5 LCDRA3
AAWDDSLNGHVV

SEQ ID NO: 44 - protein, 5 aa, Artificial Sequence
  scFv5 HCDRA1
SYWIG

SEQ ID NO: 45 - protein, 17 aa, Artificial Sequence
  scFv5 HCDRA2
IIYPGDSDTRYSPSFQG

SEQ ID NO: 46 - protein, 8 aa, Artificial Sequence
  scFv5 HCDRA3
GIYDAFDI

SEQ ID NO: 47 - protein, 12 aa, Artificial Sequence
  scFv6 LCDRA1
RASQSVDSNFLA

SEQ ID NO: 48 - protein, 7 aa, Artificial Sequence
  scFv6 LCDRA2
GVSSSAT

SEQ ID NO: 49 - protein, 8 aa, Artificial Sequence
  scFv6 LCDRA3
QQYGSSPT

SEQ ID NO: 50 - protein, 5 aa, Artificial Sequence
  scFv6 HCDRA1
DYAMH

SEQ ID NO: 51 - protein, 17 aa, Artificial Sequence
  scFv6 HCDRA2
GISWNSGSIGYADSVKG

SEQ ID NO: 52 - protein, 9 aa, Artificial Sequence
  scFv6 HCDRA3
SGDYGAFDI

SEQ ID NO: 53 - protein, 13 aa, Artificial Sequence
  scFv7 LCDRA1
SGSSSNIGNNYVS

SEQ ID NO: 54 - protein, 7 aa, Artificial Sequence
  scFv7 LCDRA2
DNNKRPS

SEQ ID NO: 55 - protein, 11 aa, Artificial Sequence
  scFv7 LCDRA3
GTWDSSLSAGV

SEQ ID NO: 56 - protein, 5 aa, Artificial Sequence
  scFv7 HCDRA1
DYAMH

SEQ ID NO: 57 - protein, 17 aa, Artificial Sequence
  scFv7 HCDRA2
GISWNSGSIGYADSVKG

SEQ ID NO: 58 - protein, 11 aa, Artificial Sequence
  scFv7 HCDRA3
EGDSSGWSPDY

SEQ ID NO: 59 - protein, 109 aa, Artificial Sequence
  scFv1 Light chain variable region
SYELTQPPSVSVAPGKTARITCGGNNIGSKSVHWYQQKPGQAPVLVVYDDSDRPSGIPERFSGSNSGNTATLTISRVEAGDEADYYCQVWDSSSDHVVFGGGTKLTVLG

SEQ ID NO: 60 - protein, 120 aa, Artificial Sequence
  scFv1 heavy chain variable region
QVQLQESGGGLVQPGRSLRLSCAASGFTFDDYAMHWVRQAPGKGLEWVSGISWNSGSIGYADSVKGRFTISRDNAKNSLYLQMNSLRAEDTALYYCAKVSAYSSSFDYWGQGTLVTVSSA

SEQ ID NO: 61 - protein, 111 aa, Artificial Sequence
  scFv2 Light chain variable region
QSALTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLG

SEQ ID NO: 62 - protein, 118 aa, Artificial Sequence
  scFv2 heavy chain variable region
QVQLLESGAEVKKPGASVKVSCKASGYTFTAYYMHWVRQAPGQGLEWMAMIDPSGSITSYAQKFQGRVTMSRDTSTSTLYMELSSLRSDDTAVYYCARDLKEGFSVPGAFDIWGQGTM

SEQ ID NO: 63 - protein, 111 aa, Artificial Sequence
  scFv3 Light chain variable region
QSVLTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLG

SEQ ID NO: 64 - protein, 125 aa, Artificial Sequence
  scFv3 heavy chain variable region
EVQLVQSGGGVAQPGRSLRLSCAASGITFNSYGMHWVRQAPGKGLEWVAVMPYDGSNEYYADSVKGRFTISRDNSKNTLYLQMNSLRAEDTAVYYCAKGSGMVQLWADAFDVWGQGTMVTVSSAS

SEQ ID NO: 65 - protein, 111 aa, Artificial Sequence
  scFv4 Light chain variable region
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVFGGGTKLTVLG

SEQ ID NO: 66 - protein, 119 aa, Artificial Sequence
  scFv4 heavy chain variable region
QVQLVQSGAEVKKPGSSVKVSCKASGGTFSSYATSWVRQAPGQGLEWMGGIIPTFGTANYAQKFQGRVTITADESTSTAYMELSSLRSEDTAVYYCAREGLGYFDYWGQGTLVTVSSAS

SEQ ID NO: 67 - protein, 112 aa, Artificial Sequence
  scFv5 Light chain variable region
QSALTQPPSASGTPGQRVTVSCSGSSSNIGSNTVNWYQQLPGTAPKLLIYSNNQRPSGVPDRFSASKSGTSASLAISGLQSEDEADYYCAAWDDSLNGHVVFGGGTKLTVLG

SEQ ID NO: 68 - protein, 119 aa, Artificial Sequence
  scFv5 heavy chain variable region
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCASGIYDAFDIWGQGTMVTVSSAS

SEQ ID NO: 69 - protein, 108 aa, Artificial Sequence
  scFv6 Light chain variable region
EIVMTQSPGTLSLSPGERATLSCRASQSVDSNFLAWYQQRPGQAPRLLIYGVSSSATGIPDRFSGSGSGTDFTLSIDRLEPEDFAVYYCQQYGSSPTFGGGTKLEIKR

SEQ ID NO: 70 - protein, 120 aa, Artificial Sequence
  scFv6 heavy chain variable region
QVQLQESGGGLVQPGRSLRLSCAASGFTFDDYAMHWVRQAPGKGLEWVSGISWNSGSIGYADSVKGRFTISRDNAKNSLYLQMNSLRAEDTAVYYCARSGDYGAFDIWGQGTMVTVSSAS

SEQ ID NO: 71 - protein, 111 aa, Artificial Sequence
  scFv7 Light chain variable region
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTKLTVLG

SEQ ID NO: 72 - protein, 122 aa, Artificial Sequence
  scFv7 heavy chain variable region
EVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKEGDSSGWSPDYWGQGTLVTVSSAS

(19) United States
(12) Patent Application Publication
(10) Pub. No.: US 2011/0311517 A1
(43) Pub. Date: Dec. 22, 2011

(54) Antibodies and methods for treating estrogen receptor-associated diseases

(75) Inventors: Jin Li, Pawcatuck, CT (US); Kun Meng, Hong Kong (CN)

(73) Assignee: SHENOGEN PHARMA GROUP, LTD., Hong Kong (CN)

(21) Appl. No.: 13/148,669

(22) PCT Filed: Feb. 10, 2010
(86) PCT No.: PCT/CN2010/070616

§ 371 Date: Aug. 10, 2011

Related U.S. Application Data
(60) Provisional application No. 61/154,753, filed on Feb. 23, 2009.

(30) Foreign Application Priority Data
Feb. 10, 2009 (CN) 200910008855.8

(51) Int. Cl. A61K 39/395; C07K 16/46; C07K 17/02; C12N 15/13; C12N 15/63; C12P 21/08; C12N 1/15; C12N 1/19; C12N 5/10; C12N 1/21; A61K 47/48; C12N 5/00; A61P 19/08; A61P 3/06; A61P 9/00; A61P 9/10; A61P 15/00; A61P 25/24; A61P 37/04; A61P 35/00; A61P 29/00; A61P 11/06; G01N 33/566; C07K 16/00

(52) U.S. Cl. 424/130.1; 530/387.1; 530/388.1; 530/387.3; 530/391.1; 536/23.53; 435/320.1; 435/69.6; 435/252.33; 435/254.11; 435/254.2; 435/254.3; 435/326; 435/252.31; 424/178.1; 435/375; 435/7.92; 436/501

(57) ABSTRACT

Provided herein in certain embodiments are antibodies, antibody fragments, pharmaceutical compositions, methods for modulating the functions of estrogen receptor alpha 36, and methods for preventing and/or treating diseases mediated by estrogen receptor alpha 36.

### FIELD OF THE INVENTION

The present invention relates to antibodies, pharmaceutical compositions and methods thereof for preventing and/or treating estrogen receptor-associated diseases.

### BACKGROUND

Estrogens are a group of hormones that are involved in many critical physiological functions in the human body. Estrogen functions include developing the female sex organs, preparing the breast and uterus for pregnancy and breast feeding after childbirth. Estrogens also play important roles in maintaining proper cardiovascular function and bone density. Estrogens are known to stimulate cell proliferation and may increase a woman's risk of developing cancers, especially breast cancer and uterus cancer.

Estrogens bind to estrogen receptors in target cells to regulate cell functions. Two types of estrogen receptors were discovered in human cells (hERs), hER-α and hER-β. They share common protein structures, each possessing three independent but interacting functional domains: the N-terminal domain (NB domain), the central DNA-binding domain (C domain), and the C-terminal ligand-binding domain (D/E/F domain). The N-terminal domain has a ligand-independent activation function (AF-1), which is involved in interaction with co-activators and transcriptional activation of target genes in the absence of ligands. The DNA binding-domain plays important roles in receptor dimerization and binding to specific DNA sequences. The C-terminal ligand binding-domain mediates ligand binding and has a ligand-dependent transactivation function (AF-2), activating gene transcription in the presence of ligands.

The full-length hER-α was identified as a 66 kDa protein and referred to as hER-α66. hER-α66 contains all three functional domains. A splice variant of hER-α66 was later discovered and named hER-α46. hER-α46 has a molecular weight of about 46 KDa and lacks the N-terminal AF-1 domain of hER-α66. Recently, a novel 36 kDa hER-α variant, hER-α36, was identified. It lacks the N-terminal AF-1 domain and the C-terminal AF-2 domain of hER-α66 (Wang et al., Biochem. Biophys. Res. Commun. 336, 1023-1027 (2005)).

hER-α66 is believed to mediate estrogen-stimulated cell proliferation via transcriptional activation of its target genes. Binding of estrogen to hER-α66 activates the transactivation domain of hER-α66 and thus stimulates the expression of downstream target genes and eventually leads to cell proliferation. hER-α46 was found to mediate membrane-initiated and estrogen-stimulated rapid NO synthesis (Li et al., Proc. Natl. Acad. Sci. USA 100: 4807-4812 (2003)). It was also shown that hER-α46, that lacks the AF-1 domain, inhibits the AF-1 activity of hER-α66 (Flouriot, G., EMBO, 19, 4688-4700, (2000)). Since hER-α36 lacks both the AF-1 and AF-2 transcriptional activation domains, it functions as a dominant-negative inhibitor of hER-α66 and hER-β to inhibit both AF-1 and AF-2 functions of hER-α and hER-β. In addition, hER-α36 is localized primarily on the plasma membrane and mediates membrane-initiated mitogenic estrogen signaling that stimulates cell proliferation. (Wang et al., Biochem. Biophys. Res. Commun. 336, 1023-1027 (2005); Wang et al., Proc. Natl. Acad. Sci. U.S.A. 103: 9063-9068 (2006)).

Extensive studies have shown that estrogen signaling is mediated via the classic nuclear transcriptional activation pathways as well as the non-classic membrane-initiated signaling pathways. It seems that hER-α66 and hER-α46 function primarily in the nucleus while hER-α36 functions mainly through outside of the nucleus

It was also shown that hER-α36 lacks Helix 8-12 of the ligand-binding domain of the original hER-α66, which totally changes the ligand binding specificity of hER-α36. Thus, hER-α36 may bind to different ligands from hER-α66 and hER-β.

As estrogen and estrogen receptor related diseases continue to affect many individuals, there remains an urgent need to discover novel approaches such as novel antibodies and methods useful to prevent and/or treat such diseases.

### SUMMARY

Provided herein are antibodies and antigen-binding fragments thereof, and pharmaceutical compositions and methods of use for treating/preventing/diagnosing conditions associated with estrogen receptor ER-α36 (SEQ ID NO. 1, Gene Accession Number BX640939).

In certain embodiments, an antibody or antigen-binding fragment provided herein specifically binds to ER-α36 but not to ER-α66 (FIG. 1(a) for ER-α66 amino acid sequence) or ER-α46 (FIG. 1(b) for ER-α46 amino acid sequence). In certain embodiments, the antibody or the antigen-binding fragment specifically binds to amino acids residues from 284 to 310 of SEQ. ID. NO: 1, or amino acid residues from 1 to 27 of SEQ. ID NO: 2.

In certain embodiments, an antibody or antigen-binding fragment provided herein substantially binds to the same epitope to which ScFv 1 (SEQ ID NO: 3), ScFv 2 (SEQ ID NO: 5), ScFv 3 (SEQ ID NO: 7), ScFv 4 (SEQ ID NO: 9), ScFv 5 (SEQ ID NO: 11), ScFv 6 (SEQ ID NO: 13), or ScFv 7 (SEQ ID NO: 15) specifically binds.

In certain embodiments, an antibody or antigen-binding fragment is provided that comprises the HCDR3, HCDR1, and/or HCDR2 sequence of ScFv 1, ScFv 2, ScFv 3, ScFv 4, ScFv 5, ScFv 6, or ScFv 7. In certain of these embodiments the antibody or antigen-binding fragment comprises the HCDR1, HCDR2, and HCDR3 sequences of ScFv 1, ScFv 2, ScFv 3, ScFv 4, ScFv 5, ScFv 6, or ScFv 7. In certain of these embodiments the antibody or antigen-binding fragment comprises the heavy chain variable region of ScFv 1, ScFv 2, ScFv 3, ScFv 4, ScFv 5, ScFv 6, or ScFv 7.

In certain embodiments, an antibody or antigen-binding comprises the LCDR1, LCDR2, and/or LCDR3 sequence of ScFv 1, ScFv 2, ScFv 3, ScFv 4, ScFv 5, ScFv 6, or ScFv 7. In certain of these embodiments, the antibody or antigen-binding further comprises the LCDR1, LCDR2, and LCDR3 sequences of ScFv 1, ScFv 2, ScFv 3, ScFv 4, ScFv 5, ScFv 6, or ScFv 7. In certain of these embodiments the antibody or antigen-binding fragment comprises the light chain variable region of ScFv 1, ScFv 2, ScFv 3, ScFv 4, ScFv 5, ScFv 6, or ScFv 7.

In certain embodiments, an antibody or antigen-binding fragment comprises 1) the HCDR3, HCDR1, and/or HCDR2 sequence of ScFv 1, ScFv 2, ScFv 3, ScFv 4, ScFv 5, ScFv 6, or ScFv 7; and 2) the LCDR1, LCDR2, and/or LCDR3 sequence of ScFv 1, ScFv 2, ScFv 3, ScFv 4, ScFv 5, ScFv 6, or ScFv 7. In certain of these embodiments, the antibody and antigen-binding fragment comprises 1) HCDR1, HCDR2, and HCDR3 sequences of ScFv 1, ScFv 2, ScFv 3, ScFv 4, ScFv 5, ScFv 6, or ScFv 7 and 2) the LCDR1, LCDR2, and LCDR3 sequences of ScFv 1, ScFv 2, ScFv 3, ScFv 4, ScFv 5, ScFv 6, or ScFv 7.

In certain embodiment, an antibody or antigen-binding fragment comprises a heavy chain variable region comprising 1) a heavy chain CDR1 selected from the group consisting of ScFv 1 HCDR1, ScFv 2 HCDR1, ScFv 3 HCDR1, ScFv 4 HCDR1, ScFv 5 HCDR1, ScFv 6 HCDR1, and ScFv 7 HCDR1; 2) a heavy chain CDR2 selected from the group consisting of ScFv 1 HCDR2, ScFv 2 HCDR2, ScFv 3 HCDR2, ScFv 4 HCDR2, ScFv 5 HCDR2, ScFv 6 HCDR2, and ScFv 7 HCDR2, and 3) a heavy chain CDR3 selected from the group consisting of ScFv 1 HCDR3, ScFv 2 HCDR3, ScFv 3 HCDR3, ScFv 4 HCDR3, ScFv 5 HCDR3, ScFv 6 HCDR3, and ScFv 7 HCDR3. ScFv m HCDR i means the HCDR i of ScFv m (1≦i≦3, 1≦m≦7).

In certain embodiment, an antibody or antigen-binding fragment comprises a light chain variable region comprising 1) a light chain CDR1 selected from the group consisting of ScFv 1 LCDR1, ScFv 2 LCDR1, ScFv 3 LCDR1, ScFv 4 LCDR1, ScFv 5 LCDR1, ScFv 6 LCDR1, and ScFv 7 LCDR1; 2) a light chain CDR2 selected from the group consisting of ScFv 1 LCDR2, ScFv 2 LCDR2, ScFv 3 LCDR2, ScFv 4 LCDR2, ScFv 5 LCDR2, ScFv 6 LCDR2, and ScFv 7 LCDR2, and 3) a light chain CDR3 selected from the group consisting of ScFv 1 LCDR3, ScFv 2 LCDR3, ScFv 3 LCDR3, ScFv 4 LCDR3, ScFv 5 LCDR3, ScFv 6 LCDR3, and ScFv 7 LCDR3. ScFv m LCDR i means the LCDR i of ScFv m (1≦i≦3, 1≦m≦7).

In certain embodiments, an antibody or antigen-binding fragment is provided that comprises 1) a heavy chain variable region comprising the HCDR3, HCDR1, and/or HCDR2 sequence of ScFv 1, ScFv 2, ScFv 3, ScFv 4, ScFv 5, ScFv 6, or ScFv 7, and 2) a light chain variable region comprising the LCDR1, LCDR2, and/or LCDR3 sequence of ScFv 1, ScFv 2, ScFv 3, ScFv 4, ScFv 5, ScFv 6, or ScFv 7. In certain of these embodiments, the antibody and antigen-binding fragment comprises 1) a heavy chain variable region comprising HCDR1, HCDR2, and HCDR3 sequences of ScFv 1, ScFv 2, ScFv 3, ScFv 4, ScFv 5, ScFv 6, or ScFv 7 and 2) a light chain variable region comprising the LCDR1, LCDR2, and LCDR3 sequences of ScFv 1, ScFv 2, ScFv 3, ScFv 4, ScFv 5, ScFv 6, or ScFv 7. In certain of these embodiments, the antibody or antigen-binding fragment comprises 1) a heavy chain variable region selected from the group consisting of any of the heavy chain variable regions of ScFv 1, ScFv 2, ScFv 3, ScFv 4, ScFv 5, ScFv 6, and ScFv 7, and 2) a light chain variable region selected from the group consisting of any of the of the heavy chain variable regions of ScFv 1, ScFv 2, ScFv 3, ScFv 4, ScFv 5, ScFv 6, and ScFv 7.

In certain embodiment, an antibody or antigen-binding fragment comprises: A) a heavy chain variable region comprising 1) a heavy chain CDR1 selected from the group consisting of ScFv 1 HCDR1, ScFv 2 HCDR1, ScFv 3 HCDR1, ScFv 4 HCDR1, ScFv 5 HCDR1, ScFv 6 HCDR1, and ScFv 7 HCDR1; 2) a heavy chain CDR2 selected from the group consisting of ScFv 1 HCDR2, ScFv 2 HCDR2, ScFv 3 HCDR2, ScFv 4 HCDR2, ScFv 5 HCDR2, ScFv 6 HCDR2, and ScFv 7 HCDR2, and 3) a heavy chain CDR3 selected from the group consisting of ScFv 1 HCDR3, ScFv 2 HCDR3, ScFv 3 HCDR3, ScFv 4 HCDR3, ScFv 5 HCDR3, ScFv 6 HCDR3, and ScFv 7 HCDR3; and

B) a light chain variable region comprising 1) a light chain CDR1 selected from the group consisting of ScFv 1 LCDR1, ScFv 2 LCDR1, ScFv 3 LCDR1, ScFv 4 LCDR1, ScFv 5 LCDR1, ScFv 6 LCDR1, and ScFv 7 LCDR1; 2) a light chain CDR2 selected from the group consisting of ScFv 1 LCDR2, ScFv 2 LCDR2, ScFv 3 LCDR2, ScFv 4 LCDR2, ScFv 5 LCDR2, ScFv 6 LCDR2, and ScFv 7 LCDR2, and 3) a light chain CDR3 selected from the group consisting of ScFv 1 LCDR3, ScFv 2 LCDR3, ScFv 3 LCDR3, ScFv 4 LCDR3, ScFv 5 LCDR3, ScFv 6 LCDR3, and ScFv 7 LCDR3.

In certain embodiments, an antibody or antigen-binding fragment is provided that comprises a heavy chain variable region comprising 1) one or more amino acid sequence set forth in SEQ ID NO 20, SEQ ID NO 21, and/or SEQ ID NO 22; 2) one or more amino acid sequence set forth in SEQ ID NO 26, SEQ ID NO 27, and/or SEQ ID NO 28; 3) one or more amino acid sequence set forth in SEQ ID NO 32, SEQ ID NO 33, and/or SEQ ID NO 34; 4) one or more amino acid sequence set forth in SEQ ID NO 38, SEQ ID NO 39, and/or SEQ ID NO 40; 5) one or more amino acid sequence set forth in SEQ ID NO 44, SEQ ID NO 45, and/or SEQ ID NO 46; 6) one or more amino acid sequence set forth in SEQ ID NO 50, SEQ ID NO 51, and/or SEQ ID NO 52; or 7) one or more amino acid sequence set forth in SEQ ID NO 56, SEQ ID NO 57, and/or SEQ ID NO 58. In certain of these embodiments, the antibody or antigen-binding fragment comprises a heavy chain variable region selected from the group consisting of SEQ ID NO 60, SEQ ID NO 62, SEQ ID NO 64, SEQ ID NO 66, SEQ ID NO 68, SEQ ID NO 70, and SEQ ID NO 72.

In certain embodiments, an antibody or antigen-binding fragment is provided that comprises a light chain variable region comprising 1) one or more amino acid sequence set forth in SEQ ID NO 17, SEQ ID NO 18, and/or SEQ ID NO 19; 2) one or more amino acid sequence set forth in SEQ ID NO 23, SEQ ID NO 24, and/or SEQ ID NO 25; 3) one or more amino acid sequence set forth in SEQ ID NO 29, SEQ ID NO 30, and/or SEQ ID NO 31; 4) one or more amino acid sequence set forth in SEQ ID NO 35, SEQ ID NO 36, and/or SEQ ID NO 37; 5) one or more amino acid sequence set forth in SEQ ID NO 41, SEQ ID NO 42, and/or SEQ ID NO 43; 6) one or more amino acid sequence set forth in SEQ ID NO 47, SEQ ID NO 48, and/or SEQ ID NO 49; or 7) one or more amino acid sequence set forth in SEQ ID NO 53, SEQ ID NO 54, and/or SEQ ID NO 55. In certain embodiments, the antibody or antigen-binding fragment comprises a light chain variable region selected from the group consisting of SEQ ID NO 59, SEQ ID NO 61, SEQ ID NO 63, SEQ ID NO 65, SEQ ID NO 67, SEQ ID NO 69, and SEQ ID NO 71.

In certain embodiments, an antibody or antigen-binding fragment is provided that comprises a heavy chain variable region comprising 1) a heavy chain CDR1 selected from the group consisting of SEQ ID NO: 20, SEQ ID NO: 26, SEQ ID NO: 32, SEQ ID NO: 38, and SEQ ID NO: 44; 2) a heavy chain CDR2 selected from the group consisting of SEQ ID NO: 21, SEQ ID NO: 27, SEQ ID NO: 33, SEQ ID NO: 39, and SEQ ID NO: 45; and 3) a heavy chain CDR3 selected from the group consisting of SEQ ID NO: 22, SEQ ID NO: 28, SEQ ID NO: 34, SEQ ID NO: 40, SEQ ID NO: 46, SEQ ID NO: 52, and SEQ ID NO: 58. In certain of these embodiments, the antibody or antigen-binding fragment comprises a heavy chain variable region selected from the group consisting of SEQ ID NO 60, SEQ ID NO 62, SEQ ID NO 64, SEQ ID NO 66, SEQ ID NO 68, SEQ ID NO 70, and SEQ ID NO 72.

In certain embodiments, an antibody or antigen-binding fragment is provided that comprises a light chain variable region comprising 1) a light chain CDR1 selected from the group consisting of SEQ ID NO: 17, SEQ ID NO: 23, SEQ ID NO: 35, SEQ ID NO: 47, and SEQ ID NO: 53; 2) a light chain CDR2 selected from the group consisting of SEQ ID NO: 18, SEQ ID NO: 24, SEQ ID NO: 36, SEQ ID NO: 48, and SEQ ID NO: 54; and 3) a light chain CDR3 selected from the group consisting of SEQ ID NO: 19, SEQ ID NO: 25, SEQ ID NO: 37, SEQ ID NO: 43, SEQ ID NO: 49, and SEQ ID NO: 55. In certain of these embodiments, the antibody or antigen-binding fragment comprises a light chain variable region selected from the group consisting of SEQ ID NO 59, SEQ ID NO 61, SEQ ID NO 63, SEQ ID NO 65, SEQ ID NO 67, SEQ ID NO 69, and SEQ ID NO 71.

In certain embodiments, an antibody or antigen-binding fragment is provided that comprises:

A) a light chain variable region comprising 1) one or more amino acid sequence set forth in SEQ ID NO 17, SEQ ID NO 18, and/or SEQ ID NO 19; 2) one or more amino acid sequence set forth in SEQ ID NO 23, SEQ ID NO 24, and/or SEQ ID NO 25; 3) one or more amino acid sequence set forth in SEQ ID NO 29, SEQ ID NO 30, and/or SEQ ID NO 31; 4) one or more amino acid sequence set forth in SEQ ID NO 35, SEQ ID NO 36, and/or SEQ ID NO 37; 5) one or more amino acid sequence set forth in SEQ ID NO 41, SEQ ID NO 42, and/or SEQ ID NO 43; 6) one or more amino acid sequence set forth in SEQ ID NO 47, SEQ ID NO 48, and/or SEQ ID NO 49; or 7) one or more amino acid sequence set forth in SEQ ID NO 53, SEQ ID NO 54, and/or SEQ ID NO 55, and

B) a heavy chain variable region comprising 1) one or more amino acid sequence set forth in SEQ ID NO 20, SEQ ID NO 21, and/or SEQ ID NO 22; 2) one or more amino acid sequence set forth in SEQ ID NO 26, SEQ ID NO 27, and/or SEQ ID NO 28; 3) one or more amino acid sequence set forth in SEQ ID NO 32, SEQ ID NO 33, and/or SEQ ID NO 34; 4) one or more amino acid sequence set forth in SEQ ID NO 38, SEQ ID NO 39, and/or SEQ ID NO 40; 5) one or more amino acid sequence set forth in SEQ ID NO 44, SEQ ID NO 45, and/or SEQ ID NO 46; 6) one or more amino acid sequence set forth in SEQ ID NO 50, SEQ ID NO 51, and/or SEQ ID NO 52; or 7) one or more amino acid sequence set forth in SEQ ID NO 56, SEQ ID NO 57, and/or SEQ ID NO 58.

In certain embodiments, an antibody or antigen-binding fragment is provided that comprises:

A) a heavy chain variable region comprising 1) a heavy chain CDR1 selected from the group consisting of SEQ ID NO: 20, SEQ ID NO: 26, SEQ ID NO: 32, SEQ ID NO: 38, and SEQ ID NO: 44; 2) a heavy chain CDR2 selected from the group consisting of SEQ ID NO: 21, SEQ ID NO: 27, SEQ ID NO: 33, SEQ ID NO: 39, and SEQ ID NO: 45; and 3) a heavy chain CDR3 selected from the group consisting of SEQ ID NO: 22, SEQ ID NO: 28, SEQ ID NO: 34, SEQ ID NO: 40, SEQ ID NO: 46, SEQ ID NO: 52, and SEQ ID NO: 58; and

B) a light chain variable region comprising 1) a light chain CDR1 selected from the group consisting of SEQ ID NO: 17, SEQ ID NO: 23, SEQ ID NO: 35, SEQ ID NO: 47, and SEQ ID NO: 53; 2) a light chain CDR2 selected from the group consisting of SEQ ID NO: 18, SEQ ID NO: 24, SEQ ID NO: 36, SEQ ID NO: 48, and SEQ ID NO: 54; and 3) a light chain CDR3 selected from the group consisting of SEQ ID NO: 19, SEQ ID NO: 25, SEQ ID NO: 37, SEQ ID NO: 43, SEQ ID NO: 49, and SEQ ID NO: 55.

In certain embodiments, the antibody or antigen-binding fragment comprises A) a light chain variable region selected from the group consisting of SEQ ID NO 59, SEQ ID NO 61, SEQ ID NO 63, SEQ ID NO 65, SEQ ID NO 67, SEQ ID NO 69, and SEQ ID NO 71; and B) a heavy chain variable region selected from the group consisting of SEQ ID NO 60, SEQ ID NO 62, SEQ ID NO 64, SEQ ID NO 66, SEQ ID NO 68, SEQ ID NO 70, and SEQ ID NO 72.

In certain embodiments, an antibody or antigen-binding fragment is provided that comprises a light chain variable region comprising one or more the amino acid sequences set forth in SEQ ID NO 17, SEQ ID NO 18, and SEQ ID NO 19, and a heavy chain variable region comprising one or more of the amino acid sequences set forth in SEQ ID NO 20, SEQ ID NO 21, and SEQ ID NO 22. In certain of these embodiments, the light chain variable region of the antibody or antigen-binding fragment comprises the amino acid sequence set forth in SEQ ID NO 59. In certain embodiments, the heavy chain variable region of this antibody or antigen-binding fragment comprises the amino acid sequence set forth in SEQ ID NO 60.

In certain embodiments, an antibody or antigen-binding fragment is provided that comprises a light chain variable region comprising one or more the amino acid sequences set forth in SEQ ID NO 23, SEQ ID NO 24, and SEQ ID NO 25, and a heavy chain variable region comprising one or more of the amino acid sequences set forth in SEQ ID NO 26, SEQ ID NO 27, and SEQ ID NO 28. In certain of these embodiments, the light chain variable region of the antibody or antigen-binding fragment comprises the amino acid sequence set forth in SEQ ID NO 61. In certain embodiments, the heavy chain variable region of this antibody or antigen-binding fragment comprises the amino acid sequence set forth in SEQ ID NO 62.

In certain embodiments, an antibody or antigen-binding fragment is provided that comprises a light chain variable region comprising one or more the amino acid sequences set forth in SEQ ID NO 29, SEQ ID NO 30, and SEQ ID NO 31, and a heavy chain variable region comprising one or more of the amino acid sequences set forth in SEQ ID NO 32, SEQ ID NO 33, and SEQ ID NO 34. In certain of these embodiments, the light chain variable region of the antibody or antigen-binding fragment comprises the amino acid sequence set forth in SEQ ID NO 63. In certain embodiments, the heavy chain variable region of this antibody or antigen-binding fragment comprises the amino acid sequence set forth in SEQ ID NO 64.

In certain embodiments, an antibody or antigen-binding fragment is provided that comprises a light chain variable region comprising one or more the amino acid sequences set forth in SEQ ID NO 35, SEQ ID NO 36, and SEQ ID NO 37, and a heavy chain variable region comprising one or more of the amino acid sequences set forth in SEQ ID NO 38, SEQ ID NO 39, and SEQ ID NO 40. In certain of these embodiments, the light chain variable region of the antibody or antigen-binding fragment comprises the amino acid sequence set forth in SEQ ID NO 65. In certain embodiments, the heavy chain variable region of this antibody or antigen-binding fragment comprises the amino acid sequence set forth in SEQ ID NO 66.

In certain embodiments, an antibody or antigen-binding fragment is provided that comprises a light chain variable region comprising one or more the amino acid sequences set forth in SEQ ID NO 41, SEQ ID NO 42, and SEQ ID NO 43, and a heavy chain variable region comprising one or more of the amino acid sequences set forth in SEQ ID NO 44, SEQ ID NO 45, and SEQ ID NO 46. In certain of these embodiments, the light chain variable region of the antibody or antigen-binding fragment comprises the amino acid sequence set forth in SEQ ID NO 67. In certain embodiments, the heavy chain variable region of this antibody or antigen-binding fragment comprises the amino acid sequence set forth in SEQ ID NO 68.

In certain embodiments, an antibody or antigen-binding fragment is provided that comprises a light chain variable region comprising one or more the amino acid sequences set forth in SEQ ID NO 47, SEQ ID NO 48, and SEQ ID NO 49, and a heavy chain variable region comprising one or more of the amino acid sequences set forth in SEQ ID NO 50, SEQ ID NO 51, and SEQ ID NO 52. In certain of these embodiments, the light chain variable region of the antibody or antigen-binding fragment comprises the amino acid sequence set forth in SEQ ID NO 69. In certain embodiments, the heavy chain variable region of this antibody or antigen-binding fragment comprises the amino acid sequence set forth in SEQ ID NO 70.

In certain embodiments, an antibody or antigen-binding fragment is provided that comprises a light chain variable region comprising one or more the amino acid sequences set forth in SEQ ID NO 53, SEQ ID NO 54, and SEQ ID NO 55, and a heavy chain variable region comprising one or more of the amino acid sequences set forth in SEQ ID NO 56, SEQ ID NO 57, and SEQ ID NO 58. In certain of these embodiments, the light chain variable region of the antibody or antigen-binding fragment comprises the amino acid sequence set forth in SEQ ID NO 71. In certain embodiments, the heavy chain variable region of this antibody or antigen-binding fragment comprises the amino acid sequence set forth in SEQ ID NO 72.

In certain of the above embodiments, the antibodies or antigen-binding fragments disclosed herein further comprise a λ light chain, a κ light chain, a γ1 heavy chain, a γ2 heavy chain, a γ3 heavy chain, or a γ4 heavy chain constant region. In certain of these embodiments, the antibodies or antigen-binding fragments comprise an IgG2 constant region. In certain embodiments, the antibodies disclosed herein are full antibodies. In certain of these embodiments, the antibody may be a monoclonal antibody, polyclonal antibody, recombinant antibody, bispecific antibody, humanized antibody, chimeric antibody, labeled antibody, bivalent antibody, anti-idiotypic antibody, or fully human antibody. In certain embodiments, an antibody or antigen-binding fragment as provided herein may be a camelized single domain antibody, a diabody, a scFv, a scFv dimer, a BsFv, a dsFv, a (dsFv)₂, a dsFv-dsFv′, an Fv fragment, a Fab, a Fab′, a F(ab′)₂, a ds diabody, a nanobody, a domain antibody, or a bivalent domain antibody.

In certain embodiments, an antibody or antigen-binding fragment is provided that specifically binds ER-α36 and/or modulates the activities of ER-α36. In certain embodiments, the antibody or antigen-binding fragment disclosed herein treats, inhibits, reduces or prevents diseases associated with ER-α36. For example, the antibody or antigen-binding fragment disclosed herein inhibits tumor growth as a function of percent tumor growth inhibition; reduces tumor size, or delays tumor growth to a specified size.

In certain embodiments, the antibodies or antigen-binding fragments bind ER-α36 with a K_{D} of ≦1000 pM. In certain of these embodiments, the antibodies or antigen-binding fragments bind ER-α36 with a K_{D} of ≦500 pM, in other embodiments 200 pM, ≦100 pM, ≦50 pM, ≦20 pM, ≦10 pM, or ≦1 pM.

In certain embodiments, methods are provided for inhibiting, treating, reducing or prevent diseases associated with ER-α36 in a subject in need thereof by administering to said subject a therapeutically effective amount of one or more antibodies or antigen-binding fragments disclosed herein. In certain embodiments, the antibody or antigen-binding fragment is administered at a dosage of about 0.01 mg/kg to about 100 mg/kg (e.g., about 10 mg.kg or about 5 mg/kg or less) per administration. In certain of these embodiments, the antibody or antigen-binding fragment is administered at a dosage of about 1 mg/kg or less per administration, in other embodiments about 0.5 mg/kg or less, and in still other embodiments about 0.1 mg/kg or less. In certain embodiments, the antibody or antigen-binding fragment is administered to the subject multiple times at an interval of once a day to once every two months. In certain of these embodiments, the antibody or antigen-binding fragment may be administered about once a week, about once every two weeks, about once a month, or about once every two months.

In certain embodiments, diagnostic methods are provided for determining the presence of ER-α36 protein or the progress/recession of a disease associated with ER-α36 by exposing a sample to the antibodies or antigen-binding fragments provided herein and determining the binding of the antibodies or antigen-binding fragments to the sample. For example, a kit is provided comprising one or more antibodies or antigen-binding fragments as disclosed herein. In certain embodiments, the kit further comprises instructions for using the antibodies or antigen-binding fragments, and/or for utilizing other components of the kit.

In certain embodiments, polynucleotides are provided that encode the amino acid sequences of the antibodies or antigen-binding fragments disclosed herein In certain other embodiments, vectors are provided that comprise these polynucleotides, and in certain other embodiments, host cells are provided that comprises these vectors. In certain embodiments, methods are provided for expressing one or more of the antibodies or antigen-binding fragments disclosed herein by culturing these host cells under conditions in which polynucleotides encoding the antibodies or antigen-binding fragments are expressed from a vector. In certain embodiments, the polynucleotides provided herein are operably associated with a promoter such as a CMV promoter in a vector. In certain embodiments, host cells comprising the vectors provided herein are Chinese hamster ovary cell.

In certain embodiments, pharmaceutical compositions are provided that comprise one or more antibodies or antigen-binding fragments as disclosed herein. In certain of these embodiments, the composition further comprises one or more pharmaceutical carriers. In certain of these embodiments, the one or more pharmaceutical carriers may be one or more pharmaceutically acceptable carriers including for example, diluents, antioxidants, adjuvants, excipients, or non-toxic auxiliary substances.

In certain embodiments, the use of one or more antibodies or antigen-binding fragments as provided herein in the manufacture of a medicament for treating a disease associated with ER-α36.

### BRIEF DESCRIPTION OF THE FIGURES

FIG. 1 shows amino acid sequences of human ERs. FIG. 1(a) shows the amino acid sequence of human ER-α66. FIG. 1(b) shows the amino acid sequence of human ER-α46.

FIG. 2 shows the presence ScFv1 (S14), ScFv2 (S24), ScFv3 (S33), ScFv4 (S41), ScFv 6 (S66) and ScFv7 (S72) in inclusion bodies via SDS-PAGE electrophoresis. FIGS. 2a, 2b, 2c, 2d, 2e, and 2f shows SDS-PAGE electrophoresis results and the presence ScFv1, ScFv2, ScFv3, ScFv4, ScFv 6 and ScFv7 in supernatants S14, S24, S33, S41, S66, and S72 respectively (See the arrows).

FIG. 3 shows the SDS-PAGE electrophoresis images of purified ScFv. FIGS. 3a, 3b, and 3c shows purified ScFv1, purified ScFv4, and purified ScFv6 respectively.

FIG. 4 shows electrophoresis images of re-natured ScFvs. In FIG. 4a Lane 1: reduced SDS-PAGE electrophoresis image of re-natured ScFv1; Lane 2: non-reduced SDS-PAGE electrophoresis image of ScFv1. FIG. 4b shows reduced SDS-PAGE electrophoresis image of renatured ScFv4 (lane 1: re-natured ScFv4; lane 2: protein marker). FIG. 4c shows reduced SDS-PAGE electrophoresis image of renatured ScFv6 (lane 1: re-natured ScFv6; lane 2: protein marker).

FIG. 5 shows reduced SDS-PAGE electrophoresis image of renatured ScFv1-ScFv7, wherein S14, S24, S33, S41, S53, S66 and S72 correspond to ScFv1, ScFv2, ScFv3, ScFv4, ScFv5, ScFv6 and ScFv7 respectively.

FIG. 6 shows ELISA results of anti-ERα36 ScFv1-ScFv7 depicting the binding affinity of ScFv1-ScFv7 with ERα36.

FIG. 7 shows the changes in relative tumor volume after tumor-bearing nude mice were administered with anti-ERα36 ScFv1, ScFv3, ScFv4, ScFv7, anti-ERα36 polyclonal antibody, negative control IgG and positive control Herceptin.

FIG. 8 shows the weight of tumors after tumor-bearing nude mice were administered with anti-ERα 36 ScFv1, ScFv3, ScFv4, ScFv7, anti-ERα36 polyclonal antibody, negative control IgG and positive control Herceptin.

FIG. 9 shows tumor inhibition rate after tumor-bearing nude mice were administered with anti-ERα36 ScFv1, ScFv3, ScFv4, ScFv7, anti-ERα36 polyclonal antibody, negative control IgG and positive control Herceptin.

FIG. 10 shows photographs of tumors obtained from tumor-bearing nude mice were treated with negative control IgG (10a), positive control Herceptin (10b), anti-ERα36 polyclonal antibodies (10c), anti-ERα36 ScFv1 (10d), anti-ERα36 ScFv3 (10e), anti-ERα36 ScFv4 (10f) and anti-ERα36 ScFv7 (10g).

FIG. 11 shows the relative tumor volume after tumor-bearing nude mice were treated with control, ScFv-7 and Tamoxifen, respectively.

FIG. 12 shows the relative tumor growth rate after tumor-bearing nude mice were treated with ScFv-7 and Tamoxifen, respectively.

FIG. 13 shows tumor weight after tumor-bearing nude mice were treated with negative control, ScFv-7 and Tamoxifen, respectively.

FIG. 14 shows the tumor growth inhibition rate after tumor-bearing nude mice were treated with ScFv-7 and Tamoxifen, respectively.

FIG. 15 shows photographs of tumors obtained from tumor-bearing nude mice treated with tamoxifen (a), anti-tumor antibodies (b) and negative control (c).

FIG. 16 shows Western blot results depicting expression of ERα36 via ScFv1-ScFv7 in breast cancer cells Sk-BR-3 and HEK293,

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

The term “antibody” as used herein includes any monoclonal antibody, polyclonal antibody, multispecific antibody, or bispecific (bivalent) antibody that binds to a specific antigen. A complete antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ, and mammalian light chains are classified as λ or κ. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variables region in both chains generally contain three highly variable loops called the complementarity determining regions (CDRs) (light (L) chain CDRs including LCDR1, LCDR2, and LCDR3, heavy (H) chain CDRs including HCDR1, HCDR2, HCDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

An antibody or antigen-binding fragment thereof that is “bivalent” comprises two antigen-binding sites. The two antigen binding sites may bind to the same antigen, or they may each bind to a different antigen, in which case the antibody or antigen-binding fragment is characterized as “bispecific.”

The term “antigen-binding fragment” as used herein refers to an antibody fragment such as for example a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In certain embodiments, an antigen-binding fragment may comprise one or more CDRs from a particular human antibody grafted to a framework region from one or more different human antibodies.

“Fab” with regard to an antibody refers to that portion of the antibody consisting of a single light chain (both variable and constant regions) bound to the variable region and first constant region of a single heavy chain by a disulfide bond.

“Fab” refers to a Fab fragment that includes a portion of the hinge region.

“F(ab′)₂ refers to a dimer of Fab.

“Fc” with regard to an antibody refers to that portion of the antibody consisting of the second and third constant regions of a first heavy chain bound to the second and third constant regions of a second heavy chain via disulfide bonding. The Fc portion of the antibody is responsible for various effector functions such as ADCC, and CDC, but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Houston 1988).

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V_{H} domains and no light chains (Riechmann 1999; Muyldermans 2001; WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman 1993; Nguyen 2002; Nguyen 2003). The variable domain of a heavy chain antibody (VHH domain) represents the smallest known antigen-binding unit generated by adaptive immune responses (Koch-Nolte 2007).

A “nanobody” refers to an antibody fragment that consists of a VHH domain from a heavy chain antibody and two constant domains, CH2 and CH3.

“Diabodies” include small antibody fragments with two antigen-binding sites, wherein the fragments comprise a V_{H} domain connected to a V_{L} domain in the same polypeptide chain (V_{H}-V_{L} or V_{H}-V_{L}) (see, e.g., Holliger 1993; EP404097; WO93/11161). By using a linker that is too short to allow pairing between the two domains on the same chain, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent domain antibody. The two V_{H} domains of a bivalent domain antibody may target the same or different antigens.

In certain embodiments, a “(dsFv)₂” comprises three peptide chains: two V_{H} moieties linked by a peptide linker and bound by disulfide bridges to two V_{L} moieties.

In certain embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In certain embodiments, a “bispecific dsFv” or dsFv-dsFv′” comprises three peptide chains: a V_{H1}-V_{H2} moiety wherein the heavy chains are linked by a peptide linker (e.g., a long flexible linker) and bound to V_{L1} and V_{L2} moieties, respectively, via disulfide bridges, wherein each disulfide paired heavy and light chain has a different antigen specificity.

In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

The term “epitope” as used herein refers to the specific group of atoms or amino acids on an antigen to which an antibody binds. Two antibodies may bind the same epitope within an antigen if they exhibit competitive binding for the antigen. For example, if an antibody or antigen-binding fragment as disclosed herein competes with ScFv 1, ScFv 2, ScFv 3, ScFv 4, ScFv 5, ScFv 6, or ScFv 7 for ER-α36 binding, the antibody may be, but is not necessarily, considered to bind the same epitope as ScFv 1, ScFv 2, ScFv 3, ScFv 4, ScFv 5, ScFv 6, or ScFv 7.

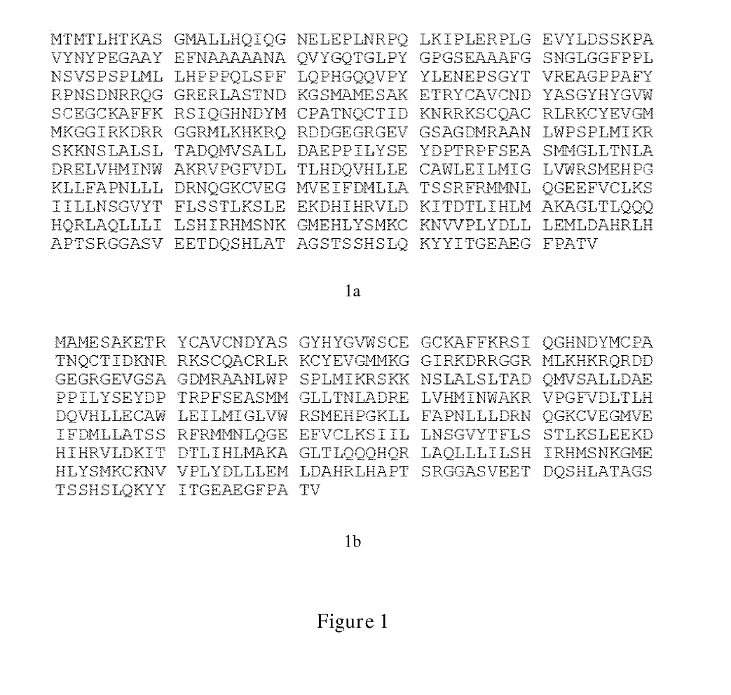

“ER” as used herein refers to one of several known estrogen receptors, ER-α66, ER-α46, or ER-α36. The full-length human ER-α identified as a 66 kDa protein having 595 amino acids is referred as hER-α66 (FIG. 1(a)) hER-α66 is composed of three independent but interacting functional domains: the N-terminal A/B domain, the C or DNA-binding domain, and the D/E/F or ligand-binding domain (See U.S. application Ser. No. 10/591,199 which is incorporated by reference herein). The N-terminal domain of ER α66 encodes a ligand-independent activation function (AF-1), a region involved in interaction with co-activators, and transcriptional activation of target genes. The DNA-binding domain or C domain contains a two zinc-finger structure, which plays an important role in receptor dimerization and binding to specific DNA sequences. The C-terminal D/E/F domain is a ligand-binding domain that mediates ligand binding, receptor dimerization, nuclear translocation, and a ligand-dependent transactivation function (AF-2). The relative contributions that both AF-1 and AF-2 exert on transcriptional control vary in a cell-specific and DNA promoter-specific manner (Berry et. al., EMBO J., 9:2811 (1990) and Tzukerman et. al., Mol. Endocrin., 8:21 (1994)). Human ER-α46 (hER-α46, FIG. 1(b)) is a splice variant of hER-α66, has a molecular weight of about 46 KDa containing 412 amino acids, and lacks the N-terminal AF-1 domain of hER-α66. Human ER-α36 (hER-α36 as set forth in SEQ ID NO:1) is a 36 kDa hER-α variant which lacks the N-terminal AF-1 domain and the C-terminal AF-2 domain of hER-α66 (Wang et al., Biochem. Biophys. Res. Commun. 336, 1023-1027 (2005), U.S. application Ser. No. 10/591,199, WO2005/087811). However, hER-α36 has a unique addition of 27 amino acid residues to its C-terminus when compared to hER-α66 or hER-α46. The 27 amino acid residues are amino acids residues from 284 to 310 of SEQ. ID. NO. 1, or amino acid residues from 1 to 27 of SEQ. ID NO. 2.

“ER activities” as used herein includes intracellular events induced by ER (e.g., hER-α36), such as receptor phosphorylation (e.g., tyrosine phosphorylation), binding of intracellular signaling molecules to the receptor or to other intracellular signaling molecules, the initiation of a signaling cascade, and/or the initiation of a biological response (e.g., induction of gene expression and changes in the physiology or development (e.g., proliferation) of the cell having the ER (e.g., hER-α36)).

“Cancer” or “cancerous condition” as used herein refers to any medical condition mediated by neoplastic or malignant cell growth, proliferation, or metastasis, and includes both solid cancers and non-solid cancers such as leukemia. “Tumor” as used herein refers to a solid mass of neoplastic and/or malignant cells.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. With regard to cancer, “treating” or “treatment” may refer to inhibiting or slowing neoplastic or malignant cell growth, proliferation, or metastasis, preventing or delaying the development of neoplastic or malignant cell growth, proliferation, or metastasis, or some combination thereof. With regard to a tumor, “treating” or “treatment” includes eradicating all or part of a tumor, inhibiting or slowing tumor growth and metastasis, preventing or delaying the development of a tumor, or some combination thereof.

The term “specifically binds” as used herein refers to a non-random binding reaction between two molecules, such as for example between an antibody and an antigen. In certain embodiments, an antibody or antigen-binding fragment that specifically binds an antigen binds the antigen with a binding affinity (K_{D}) of ≦10⁻⁶ M (e.g., ≦5×10⁻⁷ M, ≦2×10⁻⁷ M, ≦10⁻⁷ M, ≦5×10⁻⁸ M, ≦2×10⁻⁸ M, ≦10⁻⁸ M, ≦5×10⁻⁹ M, ≦2×10⁻⁹ M, ≦10⁻⁹ M, 10⁻¹⁰ M). K_{D} as used herein refers to the ratio of the dissociation rate to the association rate (k_{off}/kₒₙ), may be determined using methods known in the art (e.g., using Biacore or Kinexa techniques).

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

The phrase “host cell” as used herein refers to a cell into which an exogenous polynucleotide and/or a vector has been introduced. A host cell may be selected from a variety of cell types, including for example bacterial cells such as E. coli or B. subtilis cells, fungal cells such as yeast cells or Aspergillus cells, insect cells such as Drosophila S2 or Spodoptera Sf9 cells, or animal cells such as fibroblasts, CHO cells, COS cells, NSO cells, HeLa cells, BHK cells, HEK 293 cells, or human cells.

A “disease associated with or related to ER or ER-α36” as used herein refers to any condition that is caused by, exacerbated by, or otherwise linked to increased or decreased activities of ER (e.g., ER-α36). Such conditions include cancers mediated by cells that are dependent on of ER (e.g., ER-α36) for growth, proliferation, or metastasis, diseases of the bone such as bone loss, bone fractures or osteoporosis, and inflammatory conditions such as for example rheumatoid arthritis, psoriasis, scleroderma, chronic obstructive pulmonary disease or asthma.

The ability to “block binding” or “compete for binding” as used herein refers to the ability of an antibody or antigen-binding fragment to inhibit the binding interaction between two molecules to any detectable agree. In certain embodiments, an antibody or antigen-binding fragment that blocks binding between two molecules inhibits the binding interaction between the two molecules by at least 50%. In certain embodiments, this inhibition may be greater than 60%, in certain embodiments greater than 70%, in certain embodiments greater than 80%, and in certain embodiments greater than 90%. In certain embodiments, the binding interaction being inhibited may be that of ScFv 1, ScFv 2, ScFv 3, ScFv 4, ScFv 5, ScFv 6, or ScFv 7 to hER-α36.

The term “therapeutically effective amount” or “effective dosage” as used herein refers to the dosage or concentration of a drug effective to treat a disease or condition associated with hER-α36. For example, with regard to the use of the antibodies or antigen-binding fragments disclosed herein to treat cancer, a therapeutically effective amount is the dosage or concentration of the antibody or antigen-binding fragment capable of eradicating all or part of a tumor, inhibiting or slowing tumor growth, inhibiting growth or proliferation of cells mediating a cancerous condition, inhibiting tumor cell metastasis, ameliorating any symptom or marker associated with a tumor or cancerous condition, preventing or delaying the development of a tumor or cancerous condition, or some combination thereof.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

Provided herein are anti-hER-α36 antibodies and antigen-binding fragments thereof that have been characterized as specifically binding to the amino acid residues 284-310 of SEQ ID NO:1 and/or possessing anti-tumor activity in vivo.

Disclosed herein are the fully human single chain antibodies ScFv 1, ScFv 2, ScFv 3, ScFv 4, ScFv 5, ScFv 6, or ScFv 7, all of which specifically bind hER-α36 (e.g., the amino acid residues 284-310 of hER-α36) and are identified by panning a phage display scFv library with a target peptide set forth in SEQ ID NO:2 which corresponds to the amino acid residues 284-310 of SEQ ID NO:1.

The amino acids sequences of SEQ ID NO:1, SEQ ID NO:2, and seven single chain antibodies are listed below (The linker peptides are underlined, all CDRs are boxed):

```
The amino acid sequence of SEQ ID NO: 1
                                Met Ala Met Glu Ser Ala Lys Glu Thr Arg Tyr Cys Ala Val Cys Asn
Asp Tyr Ala Ser Gly Tyr His Tyr Gly Val Trp Ser Cys Glu Gly Cys Lys Ala Phe Phe
Lys Arg Ser Ile Gln Gly His Asn Asp Tyr Met Cys Pro Ala Thr Asn Gln Cys Thr Ile
Asp Lys Asn Arg Arg Lys Ser Cys Gln Ala Cys Arg Leu Arg Lys Cys Tyr Glu Val Gly
Met Met Lys Gly Gly Ile Arg Lys Asp Arg Arg Gly Gly Arg Met Leu Lys His Lys Arg
Gln Arg Asp Asp Gly Glu Gly Arg Gly Glu Val Gly Ser Ala Gly Asp Met Arg Ala Ala
Asn Leu Trp Pro Ser Pro Leu Met Ile Lys Arg Ser Lys Lys Asn Ser Leu Ala Leu Ser
Leu Thr Ala Asp Gln Met Val Ser Ala Leu Leu Asp Ala Glu Pro Pro Ile Leu Tyr Ser
Glu Tyr Asp Pro Thr Arg Pro Phe Ser Glu Ala Ser Met Met Gly Leu Leu Thr Asn Leu
Ala Asp Arg Glu Leu Val His Met Ile Asn Trp Ala Lys Arg Val Pro Gly Phe Val Asp
Leu Thr Leu His Asp Gln Val His Leu Leu Glu Cys Ala Trp Leu Glu Ile Leu Met Ile
Gly Leu Val Trp Arg Ser Met Glu His Pro Gly Lys Leu Leu Phe Ala Pro Asn Leu Leu
Leu Asp Arg Asn Gln Gly Lys Cys Val Glu Gly Met Val Glu Ile Phe Asp Met Leu Leu
Ala Thr Ser Ser Arg Phe Arg Met Met Asn Leu Gln Gly Glu Glu Phe Val Cys Leu Lys
Ser Ile Leu Leu Leu Asn Ser Gly Ile Ser His Val Glu Ala Lys Lys Arg Ile Leu Asn
Leu His Pro Lys Ile Phe Gly Asn Lys Trp Phe Pro Arg Val
The amino acid sequence of SEQ ID NO: 2
                                Gly Ile Ser His Val Glu Ala Lys Lys Arg Ile Leu Asn Leu His Pro
Lys Ile Phe Gly Asn Lys Trp Phe Pro Arg Val
```

The amino acid sequence of ScFv1 (SEQ ID NO:3):

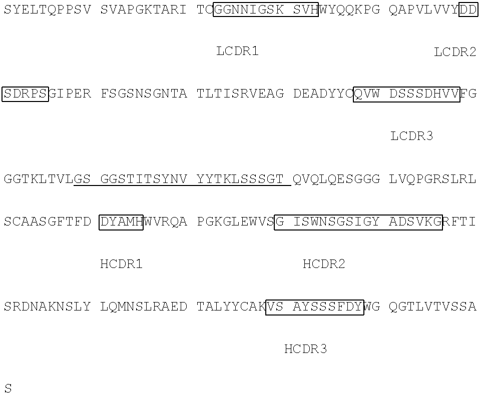

The amino acid sequence of ScFv2 (SEQ ID NO:5):

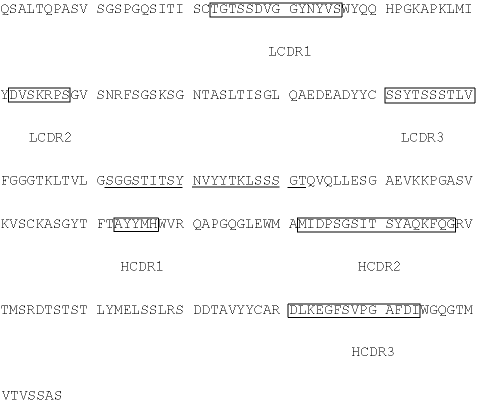

The amino acid sequence of ScFv3 (SEQ ID NO:7):

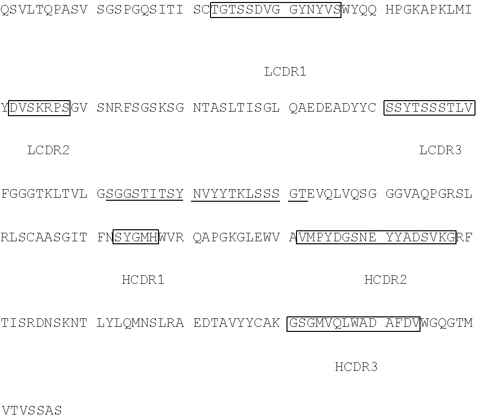

The amino acid sequence of ScFv4 (SEQ ID NO:91:

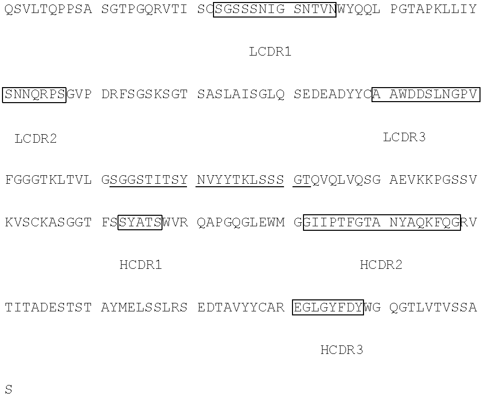

The amino acid sequence of ScFv5 (SEQ ID NO:11):

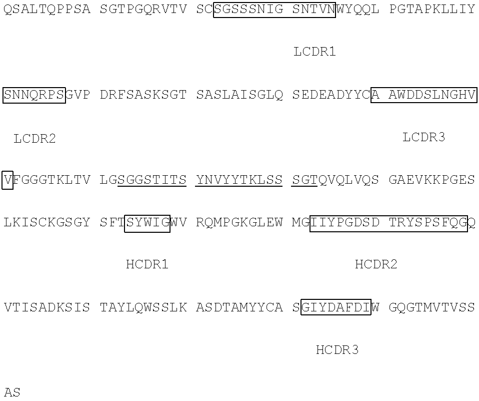

The amino acid sequence of ScFv6 (SEQ ID NO:13):

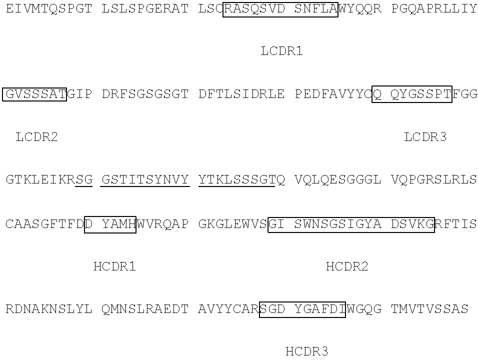

The amino acid sequences of ScFv7 (SEQ ID NO:15):

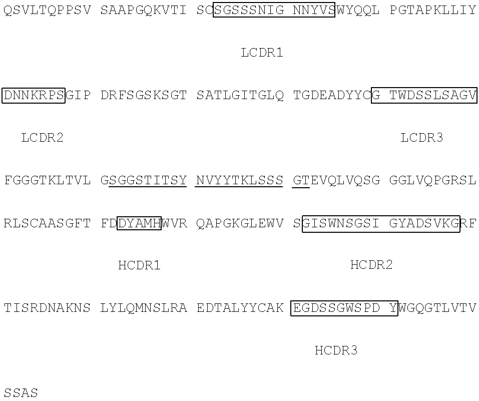

The nucleotide sequences encoding seven single chain antibodies are listed below:

The nucleotide sequences of ScFv1
(SEQ ID NO: 4)
5′-TCCTATGAGCTGACTCAGCCACCCTCGGTGTCAGTGGCCCCAGGAAAGACGGCCAGGATTACCTGTGGGG
GAAACAACATTGGAAGTAAAAGTGTGCACTGGTACCAGCAGAAGCCAGGCCAGGCCCCTGTGCTGGTCGTCTA
TGATGATAGCGACCGGCCCTCAGGGATCCCTGAGCGATTCTCTGGCTCCAACTCTGGGAACACGGCCACCCTG
ACCATCAGCAGGGTCGAAGCCGGGGATGAGGCCGACTATTACTGTCAGGTGTGGGATAGTAGTAGTGATCATG
TGGTATTCGGCGGAGGGACCAAGCTCACCGTCCTAGGTTCCGGAGGGTCGACCATAACTTCGTATAATGTATA
CTATACGAAGTTATCCTCGAGCGGTACCCAGGTGCAGCTGCAGGAGTCTGGGGGAGGCTTGGTACAGCCTGGC
AGGTCCCTGAGACTTTCCTGTGCAGCCTCTGGATTCACCTTTGATGATTATGCCATGCACTGGGTCCGGCAAG
CTCCAGGGAAGGGCCTGGAGTGGGTCTCAGGTATTAGTTGGAATAGTGGTAGCATAGGCTATGCGGACTCTGT
GAAGGGCCGATTCACCATCTCCAGAGACAACGCCAAGAACTCCCTGTATCTGCAAATGAACAGTCTGAGAGCT
GAGGACACGGCCTTGTATTACTGTGCAAAAGTATCCGCGTATAGCAGCTCGTTTGACTACTGGGGCCAGGGAA
CCCTGGTCACCGTCTCCTCAGCTAGC-3′
The nucleotide sequences of ScFv2
(SEQ ID NO: 6)
5′-CAGTCTGCCCTGACTCAGCCTGCCTCCGTGTCTGGGTCTCCTGGACAGTCGATCACCATCTCCTGCACTG
GAACCAGCAGTGACGTTGGTGGTTATAACTATGTCTCCTGGTACCAACAGCACCCAGGCAAAGCCCCCAAACT
CATGATTTATGATGTCAGTAAGCGGCCCTCAGGGGTTTCTAATCGCTTCTCTGGCTCCAAGTCTGGCAACACG
GCCTCCCTGACCATCTCTGGGCTCCAGGCTGAGGACGAGGCTGATTATTACTGCAGCTCATATACAAGCAGCA
GCACTTTGGTGTTCGGCGGAGGGACCAAGCTGACCGTCCTAGGTTCCGGAGGGTCGACCATAACTTCGTATAA
TGTATACTATACGAAGTTATCCTCGAGCGGTACCCAGGTGCAGCTGTTGGAGTCTGGGGCTGAGGTGAAGAAG
CCCGGGGCCTCAGTGAAGGTTTCCTGCAAGGCATCTGGATACACCTTCACCGCCTACTATATGCACTGGGTGC
GACAGGCCCCTGGACAAGGGCTTGAGTGGATGGCAATGATCGACCCCAGTGGTAGTATCACAAGCTACGCACA
GAAGTTCCAGGGCAGAGTCACCATGAGCAGGGACACGTCCACGAGCACACTCTACATGGAGCTGAGCAGCCTG
AGATCTGACGACACGGCCGTGTATTACTGTGCGAGAGATCTGAAAGAGGGGTTTAGTGTCCCTGGGGCTTTTG
ATATCTGGGGCCAAGGGACAATGGTCACTGTCTCTTCAGCTAGC-3′
The nucleotide sequences of ScFv3
(SEQ ID NO: 8)
5′-CAGTCTGTGTTGACGCACCCTGCCTCCGTGTCTGGGTCTCCTGGACAGTCGATCACCATCTCCTGCACTG
GAACCAGCAGTGACGTTGGTGGTTATAACTATGTCTCCTGGTACCAACAGCACCCAGGCAAAGCCCCCAAACT
CATGATTTATGATGTCAGTAAGCGGCCCTCAGGGGTTTCTAATCGCTTCTCTGGCTCCAAGTCTGGCAACACG
GCCTCCCTGACCATCTCTGGGCTCCAGGCTGAGGACGAGGCTGATTATTACTGCAGCTCATATACAAGCAGCA
GCACTCTGGTATTCGGCGGAGGGACCAAGCTCACCGTCCTAGGTTCCGGAGGGTCGACCATAACTTCGTATAA
TGTATACTATACGAAGTTATCCTCGAGCGGTACCGAGGTCCAGCTGGTACAGTCTGGGGGAGGCGTGGCCCAG
CCTGGGAGGTCCCTGAGACTCTCCTGTGCAGCCTCTGGAATCACCTTCAATAGCTATGGCATGCACTGGGTCC
GCCAGGCTCCAGGCAAGGGGCTGGAGTGGGTGGCAGTTATGCCATATGATGGAAGTAATGAATACTATGCAGA
CTCCGTGAAGGGCCGATTCACCATCTCCAGAGACAATTCCAAGAACACACTGTATCTGCAAATGAACAGCCTG
AGAGCTGAGGACACGGCTGTGTATTACTGTGCGAAAGGTTCCGGGATGGTTCAGCTATGGGCGGATGCTTTTG
ATGTCTGGGGCCAAGGGACAATGGTCACCGTCTCTTCAGCTAGC-3′
The nucleotide sequences of ScFv4
(SEQ ID NO: 10)
5′-CAGTCTGTGTTGACGCAGCCACCCTCAGCGTCTGGGACCCCCGGGCAGAGGGTCACCATCTCTTGTTC
TGGAAGCAGCTCCAACATCGGAAGTAATACTGTAAACTGGTACCAGCAGCTCCCAGGAACGGCCCCCAAAC
TCCTCATCTATAGTAATAATCAGCGGCCCTCAGGGGTCCCTGACCGATTCTCTGGCTCCAAGTCTGGCACC
TCAGCCTCCCTGGCCATCAGTGGGCTCCAGTCTGAGGATGAGGCTGATTATTACTGTGCAGCATGGGATGA
CAGCCTGAATGGTCCGGTGTTCGGCGGAGGGACCAAGCTGACCGTCCTAGGTTCCGGAGGGTCGACCATAA
CTTCGTATAATGTATACTATACGAAGTTATCCTCGAGCGGTACCCAGGTCCAGCTGGTACAGTCTGGGGCT
GAGGTGAAGAAGCCTGGGTCCTCGGTGAAGGTCTCCTGCAAGGCTTCTGGAGGCACCTTCAGCAGCTATGC
TACCAGCTGGGTGCGACAGGCCCCTGGACAAGGGCTTGAGTGGATGGGAGGGATCATCCCTACCTTTGGTA
CAGCAAACTACGCACAGAAGTTCCAGGGCAGAGTCACGATTACCGCGGACGAATCCACGAGCACAGCCTAC
ATGGAGCTGAGCAGCCTGAGATCTGAGGACACGGCCGTGTATTACTGTGCGAGAGAGGGTCTGGGCTACTT
TGACTACTGGGGCCAGGGAACCCTGGTCACCGTCTCCTCAGCTAGC-3′
The nucleotide sequences of ScFv5
(SEQ ID NO: 12)
5′-CAGTCTGCCCTGACTCAGCCACCCTCAGCGTCTGGGACCCCCGGGTAGAGGGTCACCGTCTCTTGTTC
TGGAAGCAGCTCCAACATCGGAAGTAATACTGTAAACTGGTACCAGCAGCTCCCAGGAACGGCCCCCAAAC
TCCTCATCTATAGTAATAATCAGCGGCCCTCAGGGGTCCCTGACCGATTCTCTGCCTCCAAGTCTGGCACC
TCAGCCTCCCTGGCCATCAGTGGGCTCCAGTCTGAGGATGAGGCTGATTATTACTGTGCAGCATGGGATGA
TAGCCTGAATGGTCATGTGGTATTCGGCGGAGGGACCAAGCTCACCGTCCTAGGTTCCGGAGGGTCGACCA
TAACTTCGTATAATGTATACTATACGAAGTTATCCTCGAGCGGTACCCAGGTGCAGCTGGTACAGTCTGGG
GCAGAGGTGAAAAAGCCCGGGGAGTCTCTGAAGATCTCCTGTAAGGGTTCTGGATACAGCTTTACCAGCTA
CTGGATCGGCTGGGTGCGCCAGATGCCCGGGAAAGGCCTGGAGTGGATGGGGATCATCTATCCTGGTGACT
CTGATACCAGATACAGCCCGTCCTTCCAAGGCCAGGTCACCATCTCAGCCGACAAGTCCATCAGCACCGCC
TACCTGCAGTGGAGCAGCCTGAAGGCCTCGGACACCGCCATGTATTACTGTGCGAGCGGAATCTATGATGC
TTTTGATATCTGGGGCCAAGGGACAATGGTCACCGTCTCCTCAGCTAGC-3′
The nucleotide sequences of ScFv6
(SEQ ID NO: 14)
5′-GAAATTGTGATGACGCAGTCTCCCGGCACCCTGTCTTTGTCTCCAGGGGAGAGAGCCACCCTCTCCTG
CAGGGCCAGTCAGAGTGTTGACAGCAACTTCTTAGCCTGGTATCAGCAAAGACCTGGCCAGGCTCCCCGGC
TCCTCATCTATGGTGTATCCAGCAGCGCCACTGGCATCCCAGACAGGTTCAGTGGCAGTGGGTCTGGGACA
GACTTCACTCTCTCCATCGACAGACTGGAGCCTGAAGATTTTGCTGTGTATTACTGTCAGCAATATGGTAG
CTCACCCACTTTCGGCGGAGGGACCAAGCTGGAAATCAAACGTTCCGGAGGGTCGACCATAACTTCGTATA
ATGTATACTATACGAAGTTATCCTCGAGCGGTACCCAGGTGCAGCTGCAGGAGTCTGGGGGAGGCTTGGTA
CAGCCTGGCAGGTCCCTGAGACTCTCCTGTGCAGCCTCTGGATTCACCTTTGATGATTATGCCATGCACTG
GGTCCGGCAAGCTCCAGGGAAGGGCCTGGAGTGGGTCTCAGGTATTAGTTGGAATAGTGGTAGCATAGGCT
ATGCGGACTCTGTGAAGGGCCGATTCACCATCTCCAGAGACAACGCCAAGAACTCACTGTATCTGCAAATG
AACAGCCTGAGAGCCGAGGACACGGCTGTTTATTACTGTGCGAGATCCGGTGACTACGGGGCTTTTGATAT
CTGGGGCCAAGGGACAATGGTCACCGTCTCCTCAGCTAGC-3′
The nucleotide sequences of ScFv7
(SEQ ID NO: 16)
5′-CAGTCTGTGCTGACGCAGCCGCCCTCAGTGTCTGCGGCCCCAGGACAGAAGGTCACCATCTCCTGCTC
TGGAAGCAGCTCCAACATTGGGAATAATTATGTATCCTGGTACCAGCAGCTCCCAGGAACAGCCCCCAAAC
TCCTCATTTATGACAATAATAAGCGACCCTCAGGGATTCCTGACCGATTCTCTGGCTCCAAGTCTGGCACG
TCAGCCACCCTGGGCATCACCGGACTCCAGACTGGGGACGAGGCCGATTATTACTGCGGAACATGGGATAG
CAGCCTGAGTGCTGGGGTGTTCGGCGGAGGGACCAAGCTGACCGTCCTAGGTTCCGGAGGGTCGACCATAA
CTTCGTATAATGTATACTATACGAAGTTATCCTCGAGCGGTACCGAGGTGCAGCTGGTACAGTCTGGGGGA
GGCTTGGTACAGCCTGGCAGGTCCCTGAGACTCTCCTGTGCAGCCTCTGGATTCACCTTTGATGATTATGC
CATGCACTGGGTCCGGCAAGCTCCAGGGAAGGGCCTGGAGTGGGTCTCAGGTATTAGTTGGAATAGTGGTA
GCATAGGCTATGCGGACTCTGTGAAGGGCCGATTCACCATCTCCAGAGACAACGCCAAGAACTCCCTGTAT
CTGCAAATGAACAGTCTGAGAGCTGAGGACACGGCCTTGTATTACTGTGCAAAAGAGGGAGATAGCAGTGG
CTGGTCCCCTGACTACTGGGGCCAGGGAACCCTGGTCACCGTCTCTTCAGCTAGC-3′

The heavy and light chain variable region sequences of seven single chain antibodies are set forth in Table 1. The CDR region sequences of the heavy and light chain regions are set forth in Tables 2 & 3. To illustrate, the light chain variable region of ScFv1 is set forth in SEQ ID NO: 59 and the heavy chain variable region of ScFv1 is set forth in SEQ ID NO: 60. The ScFv1 light chain variable region as set forth in SEQ ID No:59 contains light chain CDR1 at residues 23-33 of SEQ ID No:59 (ScFv1 LCDR1, SEQ ID NO:17), light chain CDR2 at residues 49-55 of SEQ ID No:59 (ScFv1 LCDR2, SEQ ID NO:18), and light chain CDR3 at residues 88-98 of SEQ ID No:59 (ScFv1 LCDR3, SEQ ID NO:19). The ScFv1 heavy chain variable region as set forth in SEQ ID NO:60 contains heavy chain CDR1 at residues 31-35 of SEQ ID NO:60 (ScFv1 HCDR1, SEQ ID NO:20), heavy chain CDR2 at residues 50-66 of SEQ ID NO:60 (ScFv1 HCDR2, SEQ ID NO:21), heavy chain CDR3 at residues 99-108 of SEQ ID NO:60 (ScFv1 HCDR3, SEQ ID NO:22).

TABLE 1

| | LIGHT CHAIN VARIABLE REGION | HEAVY CHAIN VARIABLE REGION |
|---|---|---|
| SCFV1 “SEQ. | SYELTQPPSVSVAPGKTARITCGGNNIGSKSVH | QVQLQESGGGLVQPGRSLRLSCAASGFTFDDYA |
| ID. NO. 3” | WYQQKPGQAPVLWYDDSDRPSGIPERFSGSNSG | MHWVRQAPGKGLEWVSGISWNSGSIGYADSVKG |
| | NTATLTISRVEAGDEADYYCQVWDSSSDHVVFG | RFTISRDNAKNSLYLQMNSLRAEDTALYYCAKV |
| | GGTKLTVLG | SAYSSSFDYWGQGTLVTVSSA |
| | SEQ. ID. NO. 59 | SEQ. ID. NO. 60 |
| SCFV2 “SEQ. | QSALTQPASVSGSPGQSITISCTGTSSDVGGYN | QVQLLESGAEVKKPGASVKVSCKASGYTFTAYY |
| ID. NO. 5” | YVSWYQQHPGKAPKLMIYDVSKRPSGVSNRFSG | MHWVRQAPGQGLEWMAMIDPSGSITSYAQKFQG |
| | SKSGNTASLTISGLQAEDEADYYCSSYTSSSTL | RVTMSRDTSTSTLYMELSSLRSDDTAVYYCARD |
| | VFGGGTKLTVLG | LKEGFSVPGAFDIWGQGTM |
| | SEQ. ID. NO. 61 | SEQ. ID. NO. 62 |
| SCFV3 “SEQ. | QSVLTQPASVSGSPGQSITISCTGTSSDVGGYN | EVQLVQSGGGVAQPGRSLRLSCAASGITFNSYG |
| ID. NO. 7” | YVSWYQQHPGKAPKLMIYDVSKRPSGVSNRFSG | MHWVRQAPGKGLEWVAVMPYDGSNEYYADSVKG |
| | SKSGNTASLTISGLQAEDEADYYCSSYTSSSTL | RFTISRDNSKNTLYLQMNSLRAEDTAVYYCAKG |
| | VFGGGTKLTVLG | SGMVQLWADAFDVWGQGTMVTVSSAS |
| | SEQ. ID. NO. 63 | SEQ. ID. NO. 64 |
| SCFV4 “SEQ. | QSVLTQPPSASGTPGQRVTISCSGSSSNIGSNT | QVQLVQSGAEVKKPGSSVKVSCKASGGTFSSYA |
| ID. NO. 9” | VNWYQQLPGTAPKLLIYSNNQRPSGVPDRFSGS | TSWVRQAPGQGLEWMGGIIPTFGTANYAQKFQG |
| | KSGTSASLAISGLQSEDEADYYCAAWDDSLNGP | RVTITADESTSTAYMELSSLRSEDTAVYYCARE |
| | VFGGGTKLTVLG | GLGYFDYWGQGTLVTVSSAS |
| | SEQ. ID. NO. 65 | SEQ. ID. NO. 66 |
| SCFV5 “SEQ. | QSALTQPPSASGTPGQRVTVSCSGSSSNIGSNT | QVQLVQSGAEVKKPGESLKISCKGSGYSFTSYW |
| ID. NO. 11” | VNWYQQLPGTAPKLLIYSNNQRPSGVPDRFSAS | IGWVRQMPGKGLEWMGIIYPGDSDTRYSPSFQG |
| | KSGTSASLAISGLQSEDEADYYCAAWDDSLNGH | QVTISADKSISTAYLQ1NSSLKASDTAMYYCAS |
| | VVFGGGTKLTVLG | GIYDAFDIVVGQGTMVIVSSAS |
| | SEQ. ID. NO. 67 | SEQ. ID. NO. 68 |
| SCFV6 “SEQ. | EIVMTQSPGTLSLSPGERATLSCRASQSVDSNF | QVQLQESGGGLVQPGRSLRLSCAASGFTFDDYA |
| ID. NO. 13” | LAWYQQRPGQAPRLLIYGVSSSATGIPDRFSGS | MHWVRQAPGKGLEWVSGISWNSGSIGYADSVKG |
| | GSGTDFTLSIDRLEPEDFAVYYCQQYGSSPTFG | RFTISRDNAKNSLYLQMNSLRAEDTAVYYCARS |
| | GGTKLEIKR | GDYGAFDIWGQGTMVTVSSAS |
| | SEQ. ID. NO. 69 | SEQ. ID. NO. 70 |
| SCFV7 “SEQ. | QSVLTQPPSVSAAPGQKVTISCSGSSSNIGNNY | EVQLVQSGGGLVQPGRSLRLSCAASGFTFDDYA |
| ID. NO. 15” | VSWYQQLPGTAPKLLIYDNNKRPSGIPDRFSGS | MHWVRQAPGKGLEWVSGISWNSGSIGYADSVKG |
| | KSGTSATLGITGLQTGDEADYYCGTWDSSLSAG | RFTISRDNAKNSLYLQMNSLRAEDTALYYCAKE |
| | VFGGGTKLTVLG | GDSSGWSPDYWGQGTLVTVSSAS |
| | SEQ. ID. NO. 71 | SEQ. ID. NO. 72 |

TABLE 2

| | Light Chain CDRs | | |
|---|---|---|---|
| ScFv | LCDRA1 | LCDRA2 | LCDRA3 |
| ScFv1 “SEQ. ID. No. 3” | GGNNIGSKSVH | DDSDRPS | QVWDSSSDHVV |
| | SEQ. ID. No. 17 | SEQ. ID. No. 18 | SEQ. ID. No. 19 |
| ScFv2 “SEQ. ID. No. 5” | TGTSSDVGGYNYVS | DVSKRPS | SSYTSSSTLV |
| | SEQ. ID. No. 23 | SEQ. ID. No. 24 | SEQ. ID. No. 25 |
| ScFv3 “SEQ. ID. No. 7” | TGTSSDVGGYNYVS | DVSKRPS | SSYTSSSTLV |
| | SEQ. ID. No. 29 | SEQ. ID. No. 30 | SEQ. ID. No. 31 |
| ScFv4 “SEQ. ID. No. 9” | SGSSSNIGSNTVN | SNNQRPS | AAWDDSLNGPV |
| | SEQ. ID. No. 35 | SEQ. ID. No. 36 | SEQ. ID. No. 37 |
| ScFv5 “SEQ. ID. No. 11” | SGSSSNIGSNTVN | SNNQRPS | AAWDDSLNGHVV |
| | SEQ. ID. No. 41 | SEQ. ID. No. 42 | SEQ. ID. No. 43 |
| ScFv6 “SEQ. ID. No. 13” | RASQSVDSNFLA | GVSSSAT | QQYGSSPT |
| | SEQ. ID. No. 47 | SEQ. ID. No. 48 | SEQ. ID. No. 49 |
| ScFv7 “SEQ. ID. No. 15” | SGSSSNIGNNYVS | DNNKRPS | GTWDSSLSAGV |
| | SEQ. ID. No. 53 | SEQ. ID. No. 54 | SEQ. ID. No. 55 |

TABLE 3

| | Heavy Chain CDRs | | |
|---|---|---|---|
| ScFv | HCDRA1 | HCDRA2 | HCDRA3 |
| ScFv1 “SEQ. ID. No. 3” | DYAMH | GISWNSGSIGYADSVKG | VSAYSSSFDY |
| | SEQ. ID. No. 20 | SEQ. ID. No. 21 | SEQ. ID. No. 22 |
| ScFv2 “SEQ. ID. No. 5” | AYYMH | MIDPSGSITSYAQKFQG | DLKEGFSVPGAFDI |
| | SEQ. ID. No. 26 | SEQ. ID. No. 27 | SEQ. ID. No. 28 |
| ScFv3 “SEQ. ID. No. 7” | SYGMH | VMPYDGSNEYYADSVKG | GSGMVQLWADAFDV |
| | SEQ. ID. No. 32 | SEQ. ID. No. 33 | SEQ. ID. No. 34 |
| ScFv4 “SEQ. ID. No. 9” | SYATS | GIIPTFGTANYAQKFQG | EGLGYFDY |
| | SEQ. ID. No. 38 | SEQ. ID. No. 39 | SEQ. ID. No. 40 |
| ScFv5 “SEQ. ID. No. 11” | SYWIG | IIYPGDSDTRYSPSFQG | GIYDAFDI |
| | SEQ. ID. No. 44 | SEQ. ID. No. 45 | SEQ. ID. No. 46 |
| ScFv6 “SEQ. ID. No. 13” | DYAMH | GISWNSGSIGYADSVKG | SGDYGAFDI |
| | SEQ. ID. No. 50 | SEQ. ID. No. 51 | SEQ. ID. No. 52 |
| ScFv7 “SEQ. ID. No. 15” | DYAMH | GISWNSGSIGYADSVKG | EGDSSGWSPDY |
| | SEQ. ID. No. 56 | SEQ. ID. No. 57 | SEQ. ID. No. 58 |

The CDR regions, the light chain regions, and the heavy chain regions of the seven disclosed ScFvs can be grafted to other framework regions or constant regions according to methods know in the art to render a camelized single domain antibody, a diabody, a BsFv, an scFv dimer, a dsFv, a (dsFv)₂, a dsFv-dsFv′, an Fv fragment, a Fab, a Fab′, a F(ab′)₂, a ds diabody, a nanobody, a domain antibody, a bivalent domain antibody, or a full antibody. The antibodies disclosed herein can be a monoclonal antibody, a recombinant antibody, a bispecific antibody, a humanized antibody, a chimeric antibody, a labeled antibody, a bivalent antibody, an anti-idiotypic antibody, or a fully human antibody.

Antibodies or antigen-binding fragments with enhance properties (e.g., increased affinity) can be generated by random mutagenesis of the CDR regions or FR regions of the seven disclosed ScFvs and subsequent binding and functional assays. Therefore, in certain embodiments, antibodies and antigen-binding fragments are provided that comprise one or more CDR sequences of the seven disclosed ScFvs, wherein the one or more CDR sequences contain one or more amino acid substitutions, additions or deletions. Antibodies and antigen-binding fragments generated in this manner may be screened for binding to ER-α36 in order to identify antibodies with improved binding characteristics. Antibodies with favorable binding characteristics may be subjected to one or more functional assays to determine their ability to, for example, inhibit cancer cell growth or proliferation in vitro or tumor growth in vivo.

Method of Using the Antibodies and Antigen-Binding Fragments

The antibodies and antigen-binding fragments provided herein have been found to inhibit tumor growth in vivo. Therefore, the antibodies and antigen-binding fragments may be used to treat various conditions or diseases associated with ER-α36.

In certain embodiments, methods of preventing and/or treating a disease associated ER-α36 in a subject comprising administering to the subject a therapeutic effective dosage of a pharmaceutical composition comprising the antibodies or antigen-binding fragments provided herein. Examples of diseases associated with ER-α36 include without limitation bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, perimenopausal depression, post-partum depression, premenstrual syndrome, manic depression, anxiety, dementia, obsessive compulsive behavior, attention deficit disorder, sleep disorders, irritability, impulsivity, immune deficiency, auto immune diseases, anger management, multiple sclerosis and Parkinson's disease, inflammation, inflammatory condition, inflammatory bowel disease, respiratory diseases, sexual dysfunction, hypertension, retinal degeneration, asthma and cancers. Preferably, diseases related to ER-α36 include bone loss, bone fracture, osteoporosis, menopause, premenstrual syndrome, endometriosis, uterine disease, impotence, sexual dysfunctions, increased levels of LDL cholesterol, cardiovascular diseases, vascular smooth muscle cell proliferation, depression resulting from an estrogen deficiency, perimenopausal depression, post-partum depression, immune deficiency, auto immune diseases, inflammation, inflammatory condition, asthma and cancerous conditions. More preferably, diseases associated with ER-α36 include bone loss, osteoporosis, impotence, cardiovascular diseases, atherosclerosis, immune deficiency, inflammation, inflammatory condition, asthma and cancerous condition. The inflammatory condition used herein includes rheumatoid arthritis, psoriasis, scleroderma, chronic obstructive pulmonary disease, and asthma. The subject may be a mammal such as a dog, cat, cow, sheep, horse, or human, preferably a human. The required therapeutic amount for the method will vary according to the specific diseases and is readily ascertainable by one of ordinary skill in the art having benefit of the instant disclosure.

In certain embodiments, methods of preventing and/or treating a cancerous condition in a subject comprising administering to the subject a pharmaceutical composition comprising the antibodies or antigen-binding fragments provided herein. Cancerous conditions and tumor types that may be treated using the antibodies or antigen-binding fragments disclosed herein include but are not limited to carcinoma, blastoma, sarcoma, germ cell tumor, or hematological or lymphoid malignancy such as leukemia, lymphoma, or multiple myeloma. More specifically, cancerous conditions and tumor types that may be treated using the antibodies disclosed herein include but are not limited to squamous cell cancer, lung cancer (e.g., small cell lung cancer, non-small cell lung cancer (NSCLC), adenocarcinoma of the lung, or squamous cell carcinoma of the lung), cancer of the peritoneum, liver cancer (e.g., hepatocellular carcinoma/hepatoma), gastric or stomach cancer (e.g., gastrointestinal cancer), pancreatic cancer, brain tumor (e.g., glioblastoma/glioblastoma multiforme (GBM), non-glioblastoma brain tumor, or meningioma), glioma (e.g., ependymoma, astrocytoma, anaplastic astrocytoma, oligodendroglioma, or mixed glioma such as oligoastrocytoma), cervical cancer, ovarian cancer, liver cancer (e.g., hepatoblastoma, hepatocellular carcinoma/hepatoma, or hepatic carcinoma), bladder cancer (e.g., urothelial cancer), breast cancer, colon cancer, colorectal cancer, rectal cancer, endometrial or uterine carcinoma, salivary gland carcinoma, kidney or renal cancer (e.g., rhabdoid tumor of the kidney), prostate cancer, vulval cancer, penile cancer, anal cancer (e.g., anal squamous cell carcinoma), thyroid cancer, head and neck cancer (e.g., nasopharyngeal cancer), skin cancer (e.g., melanoma or squamous cell carcinoma), osteosarcoma, Ewing's sarcoma, chondrosarcoma, soft tissue sarcoma (e.g., rhabdomyosarcoma, fibrosarcoma, Kaposi's sarcoma), carcinoid cancer, eye cancer (e.g., retinoblastoma), mesothelioma, lymphocytic/lymphoblastic leukemia (e.g., acute lymphocytic/lymphoblastic leukemia (ALL) of both T-cell lineage and B-cell precursor lineage, chronic lymphoblastic/lymphocytic leukemia (CLL), acute myelogenous/myeloblastic leukemia (AML), including mast cell leukemia, chronic myelogenous/myelocytic/myeloblastic leukemia (CML), hairy cell leukemia (HCL), Hodgkin's disease, non-Hodgkin's lymphoma, chronic myelomonocytic leukemia (CMML), follicular lymphoma (FL), diffuse large B cell lymphoma (DLCL), mantle cell lymphoma (MCL), Burkitt's lymphoma (BL), mycosis fungoides, Sezary syndrome, cutaneous T-cell lymphoma, mast cell neoplasm, medulloblastoma, nephroblastoma, solitary plasmacytoma, myelodysplastic syndrome, chronic and non-chronic myeloproliferative disorder, central nervous system tumor, pituitary adenoma, vestibular schwannoma, primitive neuroectodermal tumor, ependymoma, choroid plexus papilloma, polycythemia vera, thrombocythemia, idiopathic myelofibrosis, and pediatric cancers such as pediatric sarcomas (e.g., neuroblastoma, rhabdomyosarcoma, and osteosarcoma). In addition, tumors can be malignant (e.g., cancers) or benign (e.g., hyperplasia, cyst, pseudocyst, hamartoma, and benign neoplasm).

In certain embodiments, the antibodies and antigen-binding fragment disclosed herein are modulators of ER-α36 and are useful for modulating the ER-α36 activities in cells in vitro and in vivo. In certain embodiments, the antibodies and antigen-binding fragment disclosed herein may induce cell death and/or inhibit cell proliferation.

In certain embodiments, methods of modulating the ER-α36 activities in a cell comprise exposing a cell expressing ER-α36 to the antibodies and antigen-binding fragment disclosed herein. The cells may express ER-α36 endogenously or exogenously through genetic engineering. In one embodiment, the cells express ER-α36 endogenously. In a preferred embodiment, the cells are cancer cells that express ER-α36 endogenously. Examples of cancer cells that express ER-α36 are breast cancer cells, leukemia cells, lung cancer cells, myeloma cells, prostate cancer cells, ovarian cancer cells, colon cancer cells and stomach cancer cells. In a further preferred embodiment, the cells expressing ER-α36 are breast cancer cells that express ER-α36 endogenously. Examples of breast cancer cells expressing ER-α36 are MCF7 and MDA-MB-231 cells. The expression of the endogenous ER-α36, may be increased or decreased through treatment with one or more agents. Examples of such agents are serum, E2β (17β-estradiol), Tamoxifen and ICI 182,780.

In another embodiment, the cells are altered by genetic engineering to express exogenous ER-α36. Cells expressing exogenous ER-α36 may be prepared by genetic engineering methods known to one of ordinary skill in the art (See Sambrook et al., Molecular Cloning, A Laboratory Manual (2d Ed. 1989) (Cold Spring Harbor Laboratory)). Briefly, an exogenous ER-α36 gene is prepared and inserted into an expression vector, which is transfected into a host cell, which is then grown in a culture solution suitable for expressing the exogenous ER-α36. An example of the gene sequence of human ER-α36 is disclosed in Wang et al., Biochem. Biophys. Res. Commun. 336, 1023-1027 (2005) (GenBank Accession No. BX640939). The cells expressing exogenous ER-α36 may or may not express endogenous ER-α36. The expression levels of endogenous or exogenous ER-α36 in the cells may be increased or decreased by treatment with one or more other agents. Examples of such agents are serum, E2β (17β-estradiol), Tamoxifen and ICI 182,780. The cells expressing ER-α36 may or may not express other estrogen receptors such as ER-α66, ER-α46 and ER-β.

The antibodies or antigen-binding fragments disclosed herein may be administered alone or in combination with one or more additional therapeutic means or agents. For example, the antibodies or antigen-binding fragments disclosed herein may be administered in combination with chemotherapy, radiation therapy, surgery for the treatment of cancer (e.g., tumorectomy), one or more anti-emetics or other treatments for complications arising from chemotherapy, or any other therapeutic agent for use in the treatment of cancer or any medical disorder mediated by ER-α36. In certain of these embodiments, an antibody or antigen-binding fragment as disclosed herein that is administered in combination with one or more additional therapeutic agents may be administered simultaneously with the one or more additional therapeutic agents, and in certain of these embodiments the antibody or antigen-binding fragment and the additional therapeutic agent(s) may administered as part of the same pharmaceutical composition. However, an antibody or antigen-binding fragment administered “in combination” with another therapeutic agent does not have to be administered simultaneously with or in the same composition as the agent. An antibody or antigen-binding fragment administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art. Examples of therapeutic agents include, but are not limited to, Icaritin, Tamoxifen, 17β-estradol, ICI 182,780, compounds disclosed in the U.S. patent application Ser. No. 11/877,575 filed on Oct. 23, 2007 which is incorporated herein by reference, compounds disclosed in the U.S. Pat. Appl. 60/046,255 filed on Apr. 8, 2008 which is incorporated herein by reference, cytokines, anti-VEGF antibodies (e.g., Bevacizumab or Avastin), anti-HER2 antibodies (e.g., Herceptin or trastuzumab), anti EGFR antibodies (Nimotuzamab or Erbitux), and tyrosin receptor inhibitors such as Gapatinib and Lapatinib.

Example of cytokines include but are not limited to lymphokines, monokines, human growth hormone, bovine growth hormone, parathyroid hormone, thyroxine, insulin, proinsulin, relaxin, prorelaxin, follicle stimulating hormone, thyroid stimulating hormone, luteinizing hormone, hepatic growth factor, fibroblast growth factor, prolactin, placental lactogen, tumor necrosis factor, mullerian-inhibiting substance, mouse gonadotropin-associated peptide, inhibin, activin, integrin, thrombopoietin, nerve growth factors such as NGF-β, platelet growth factor, transforming growth factors such as TGF-α and TGF-β, insulin-like growth factor I and II, erythropoietin, osteoinductive factors, interferons such as interferon-α, -β, and -γ, colony stimulating factors such as macrophage-CSF, granulocyte macrophage CSF, and granulocyte-CSF, interleukins such IL-1, IL-1α, IL-2, IL-3, IL-4, IL-5, IL-6, IL-7, IL-8, IL-9, IL-10, IL-11, and IL-12, tumor necrosis factors such as TNF-α and TNF-β, and other polypeptide factors.

In certain embodiments, antibodies or antigen-binding fragments disclosed herein are used by being linked to or in combination with one or more chemotherapeutic agents. Examples of chemotherapeutic agents include, but are not limited to, amrubicin, atrasentan batabulin, calcitriol, cilengitide, dasatinib, decatanib, edotecarin, enzastaurin, erlotinib, everolimus, gimatecan, gossypol ipilimumab, lonafarnib, lucanthone, neuradiab, nolatrexed, oblimersen, ofatumumab, oregovomab, panitumumab, pazopanibrubitecan, talampanel, temsirolimus, tesmilifene, tetrandrine, ticilimumab, trabectedin, vandetanib, vitespan, zanolimumab, zolendronate, histrelin, azacitidine, dexrazoxane, alemtuzumab, lenalidomide, gemtuzumab, ketoconazole, nitrogen mustard, ibritumomab tiuxetan, decitabine, hexamethylmelamine, bexarotene, tositumomab, arsenic trioxide, editronate, cyclosporine, Edwina-asparaginase, and strontium 89.

It is contemplated that a variety of conjugates may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the antibodies or antigen-binding fragments by covalent binding, affinity binding, intercalation, coordinate binding, complexation, association, blending, or addition, among other methods. In certain embodiments, the antibodies and antigen-binding fragments disclosed herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate. In certain embodiments, the antibodies may be linked to a conjugate indirectly, or through another conjugate. For example, the antibody or antigen-binding fragments may be conjugated to biotin, then indirectly conjugated to a second conjugate that is conjugated to avidin.

In certain embodiments, conjugates linked to the antibodies or antigen-binding fragments disclosed herein may comprise one or more agents meant to alter one or more pharmacokinetic (PK) properties of the antibody or antigen-binding fragment, such as for example polyethylene glycol (PEG) to increase the half-life or decrease the immunogenicity of the antibody or antigen-binding fragment

In certain embodiments, conjugates linked to the antibodies or antigen-binding fragments disclosed herein may comprise one or more detectable labels. Such labels include, but are not limited to, radioactive isotopes such as ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides, luminescent labels, fluorescent labels such as for example fluorescein, rhodamine, dansyl, phycoerythrin, or Texas Red, and enzyme-substrate labels such as for example horseradish peroxidase, alkaline phosphatase, or β-D-galactosidase.

In certain embodiments, the antibodies or antigen-binding fragments provided herein may be administered as part of a pharmaceutical composition that comprises one or more pharmaceutical acceptable carriers. Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, or emulsifiers.

Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The therapeutic effective dosage of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of tumor development. Dosages may be proportionally reduced or increased by one of ordinary skill in the art (e.g., physician or veterinarian) as indicated by these and other circumstances or requirements.

In certain embodiments, an antibody or antigen-binding fragment as provided herein may be administered at a therapeutically effective dosage of about 0.01 mg/kg to about 100 mg/kg (e.g., about 0.01 mg/kg, about 0.5 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg). In certain of these embodiments, the antibody or antigen-binding fragment is administered at a dosage of about 50 mg/kg or less, and in certain of these embodiments the dosage is 10 mg/kg or less, 5 mg/kg or less, 1 mg/kg or less, 0.5 mg/kg or less, or 0.1 mg/kg or less. A given dosage may be administered at various intervals, such as for example once a day, two or more times per day, two or more times per week, once per week, once every two weeks, once every three weeks, once a month, or once every two or more months. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies and antigen-binding fragments disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes. In embodiments wherein the antibodies or antigen-binding fragments are administered via injection, injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-hER antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the lyophilized powder is added to sterile and/or non-pyretic water or other liquid suitable carrier. The precise amount depends upon the selected therapy being given, and can be empirically determined.

The antibodies and antigen-binding fragments provided herein may be used in various non-therapeutic uses. In certain embodiments, the antibodies or antigen-binding fragments may be used as affinity purification agents to purify ER-α36 or fragments thereof. In these embodiments, the antibodies or antigen-binding fragments may be immobilized on a solid phase such as a resin or filter paper using methods known in the art. The antibodies or antigen-binding fragments may also be used to precipitate ER-α36 or fragments thereof from solution. In other non-therapeutic embodiments, the antibodies or antigen-binding fragments may be used in various in vitro or in vivo diagnostic or detection applications. In certain of these embodiments, the antibodies or antigen-binding fragments may be conjugated to a detectable label. In other embodiments, the antibodies or antigen-binding fragments may not be conjugated to a detectable label, but may be detected using a labeled secondary antibody that binds to the antibody. In certain embodiments, the antibodies or antigen-binding fragments disclosed herein may be used to detect ER-α36 expression. In certain of these embodiments, the antibodies or antigen-binding fragments may be used to diagnose a condition associated with increased or decreased ER-α36 expression. For example, the antibody or antigen-binding fragment may be contacted with a biological sample from a subject in order to diagnose a condition associated with increased or decreased ER-α36 expression in the subject, in particular, the progression or recession of a condition associated with ER-α36. Likewise, the antibody or antigen-binding fragment may be administered to the subject directly, with binding to ER-α36 detected using methods known in the art.

In certain embodiments, isolated nucleic acid encoding the antibodies or antigen-binding fragment herein, vectors and host cells comprising the nucleic acid, and recombinant techniques for the production of the antibody are provided.

For recombinant production of the antibody, the nucleic acid encoding it may be isolated and inserted into a replicable vector for further cloning (amplification of the DNA) or for expression. In another embodiment, the antibody may be produced by homologous recombination known in the art. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-ER antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for anti-ER antibody production and cultured in conventional nutrient media modified as appropriate for inducing promoters, selecting transformants, or amplifying the genes encoding the desired sequences.

The host cells used to produce the antibodies or antigen-binding fragments provided herein may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human .gamma.1, .gamma.2, or .gamma.4 heavy chains (Lindmark et al., J. Immunol. Meth. 62:1-13 (1983)). Protein G is recommended for all mouse isotypes and for human.gamma.3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C.sub.H3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

In certain embodiments, the antibodies or antigen-binding fragments provided herein can be provided in a kit, i.e., a packaged combination of reagents in predetermined amounts with instructions for performing the diagnostic assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

In certain embodiments, an article of manufacture containing materials useful for the treatment of the conditions described above is provided. The article of manufacture comprises a container and a label. Suitable containers include, for example, bottles, vials, syringes, and test tubes. The containers may be formed from a variety of materials such as glass or plastic. The container holds a pharmaceutical composition provided herein (comprising the antibodies or antigen-binding fragment disclosed herein) which is effective for treating the condition and may have a sterile access port (for example the container may be an intravenous solution bag or a vial having a stopper pierceable by a hypodermic injection needle). The label on or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Screening of Human Phages Displaying Anti-ERα-36 scFv

Preparation of ERα36 and Biotinylated ERα36.

ERα36 was prepared using chemical synthesis, and then labeled with biotin to obtain biotinylated ERα36.

Phage Display.

Phage display was used to screen phages displaying human anti-ERα36 scFv for its considerable advantage over the classic hybridoma technology. For example, phage display can obtain the human antibodies directly so as to avoid the humanization modification of the antibodies obtained from the hybridoma technology. Phage display is well-known in the art, and the detailed description can be found in various literatures (see for example, Phage antibodies: filamentous phage displaying antibody variable domains. Nature. 1990 Dec. 6; 348(6301):552-4.). Phage display was carried out as described below.

Preparation of a Phage Library.

A phage library was constructed using scFv expression vectors. Normal human lymph cells were separated and purified to extract total RNA which was used in cDNA synthesis. The antibody variable region genes and scFv genes were amplified using polymerase chain reaction (PCR). The PCR products of the heavy chain variable region V_{H} and the light chain variable region V_{L} were purified to prepare DNA fragments which were assembled into the scFv fragments to construct the phage display scFv library. The detailed protocol can be found in the literature. (Lennard s., Standard protocols for the construction of scFv libraries. Methods Mol. Biol. 2002; 178: 59-71; Sheets M D, Efficient high-affinity human single-chain antibodies to protein antigens, Proc. Natl. Acad. Sci. U.S.A. 1998 May 26; 95(11):6157-62.)

Panning of Phages Displaying Human Anti-ERα36 scFv.

Panning tubes and streptavidin-conjugated magnetic beads (purchased from Promega) were blocked at 4° C. overnight with blocking buffer (0.1 M NaHCO₃, pH8.6, 5 mg/ml BSA, 0.02% NaN₃, 0.1 μg/ml streptavidin). Blocked tubes and beads were washed with 0.1% PBST (PBS with 0.1% Tween 20 (v/v)). During the first round of panning process, 4×10¹² pfu phage library was mixed with equal volume of 4% PBSM (PBS containing 4% milk) and incubated at room temperature for 60 min. A final concentration of 10 μg/ml biotinylated ERα36 was added into the phage mix, and incubated at room temperature for 30 to 60 min. Streptavidin-conjugated magnetic beads/antigen samples were washed with PBST using a magnetic separator. The samples were re-suspended in 2% PBSM, and equilibrated at room temperature for 1-2 h. Equilibrated beads were separated from PBSM, re-suspended in the mixture of phages and biotinylated ERα36 peptides and incubated for 15 min at room temperature. The panning tubes were then placed in a magnetic separator and flipped up and down for 2 min. The liquid in the panning tubes was removed and the beads were washed with PBSMT (PBS containing 2% milk and a certain percent of Tween-20). The beads were transferred to new tubes to be washed by PBSMT and then transferred to new tubes to be washed by PBS. The beads were finally transferred to new tubes to elute the phages from the beads at room temperature using acidic elution buffer. The eluted phages were used in the next round of panning.

The Second Round of Panning.

The eluted phages obtained from the first round of panning were used to infect log-phase TG1 bacteria. After propagation, 4.0×10¹² pfu phages were used in the second round of panning, following the same procedure as used in the first round of panning, except that the 0.1% PBST was replaced with 0.5% PBST and the final concentration of the biotinylated ERα36 added to the phage mix was 1 μg/ml.

The Third Round of Panning.

The eluted phages obtained from the second round of panning were used to infect log-phase TG1 bacteria. After propagation, 3.9×10¹² pfu phages were used in the third round of panning, following the same procedure as used in the second round of panning, except that the final concentration of the biotinylated ERα36 added to the phages was 0.1 μg/ml.

The Fourth Round of Panning.

The eluted phages obtained in the third round of panning were used to infect log-phase TG1 bacteria. After propagation, 4.0×10¹² pfu phages were used in the fourth round of panning, following the same procedure as used in the third round of panning, except that 1 mg/ml ERα36 was used in stead of the acidic elution buffer to competitively elute the phages.

The detailed conditions and results for the four rounds of panning process are listed in Table 4. The screening stringency is remarkably improved by incubating phages with decreased concentrations of biotinylated ERα36 and increased percentage of Tween-20. To ensure a recovery of diversified binders during the first three rounds, acidic elution was used. During the last round of panning, competitive elution was accomplished using high concentration of non-biotinylated ERα36 to competitively bind to phages displaying anti-ERα36. As shown in Table 4, the enriching factors decrease effectively, demonstrating an obvious enriching effect.

TABLE 4
Summary of the panning process

| | | | | Enriching |
|---|---|---|---|---|
| Round | Conditions | Input (pfu) | Output (pfu) | factor |
| 1^{st} | Target protein: 10 μg/mL biotinylated ER36 | 4 × 10¹² | 1.35 × 10⁶ | 2.96 × 10⁶ |
| | Washing buffer: 0.1% Tween-20-PBS, 10 | | | |
| | times | | | |
| | Elution buffer: acidic elution buffer | | | |
| 2^{nd} | Target protein: 1 μg/mL biotinylated ER36 | 4.0 × 10¹² | 8.9 × 10⁶ | 4.46 × 10⁵ |
| | Washing buffer: 0.5% Tween-20-PBS, 15 | | | |
| | times | | | |
| | Elution buffer: acidic elution buffer | | | |
| 3^{rd} | Target protein: 0.1 μg/mL biotinylated ER36 | 3.9 × 10¹² | 5.5 × 10⁶ | 7.22 × 10⁵ |
| | Washing buffer: 0.5% Tween-20-PBS, 15 | | | |
| | times | | | |
| | Elution buffer: acidic elution buffer | | | |
| 4^{th} | Target protein: 0.1 μg/mL biotinylated ER36 | 4.0 × 10¹² | 2.26 × 10⁷ | 1.77 × 10⁵ |
| | Washing buffer: 0.5% Tween-20-PBS, 15 | | | |
| | times | | | |
| | Elution buffer: 1 mg/mL ER36 | | | |

Note:
Enriching factor = Input/Output.

### Example 2

### Identifying Positive Phage Clones Expressing Human Anti-ERα36 scFv

Selection of Single Clones of Anti-Body-Displaying Phages.

Phage single clones obtained from the fourth round of panning as described in Example 1 were inoculated respectively into 2TY-AG medium (2TY containing 100 μg/ml ampicillin and 1% (w/v) glucose), and incubated overnight at 37° C. 100 μl of the cultured cells were added to 20 ml 2TY-AG medium, and incubated at 37° C. until OD600 reached 0.4-0.5. Helper phages were added and cultured at 37° C. to infect the phages with bacteria. The infected bacteria were collected by centrifugation at 5000 g for 10 min, re-suspended in 2TY-Ak, and cultured at 37° C. for 16 h. The phages were precipitated by phage precipitating agents, followed by centrifugation to remove bacteria debris. Phages were re-suspended in PBS, centrifuged again to remove antibody fragments not associated with phages, and re-suspended in PBS.

Phage ELISA.

Neutravidin-coated plates (purchased from Pierce) were washed with washing buffer (PBS containing 0.1% Tween-20) and all wells of the plates were blocked by incubating in blocking buffer at 4° C. for 1-2 h. Then the blocked plates were taken away from the blocking buffer, washed six times with washing buffer and dried upside-down. Biotinylated ERα36 in PBS (100 μl) was added to each testing well of the plates, incubated for 1-2 h at room temperature and removed. The plates were washed once with washing buffer. Phage solution (100 μl) was added per well and incubated for 1-2 h at room temperature. The plates were washed six times with the washing buffer. HRP-conjugated rabbit anti-M13 antibody (purchased from GE Healthcare) was diluted by 1:5,000 in blocking buffer. The diluted HRP-conjugated rabbit anti-M13 antibody (100 μl) was added to each well and incubated for 1 h at room temperature followed by washing six times with washing buffer.

HRP substrate solution (100 μl) was added to each testing well and incubated for 30 min at room temperature. The signals were detected using a microplate reader at 490 nm. The HRP substrate solution was prepared as follows: an OPD stock solution was prepared by dissolving 22 mg OPD (purchased from Sigma) in 100 ml of sodium citrate (50 mM, pH 4) followed by filtration and sterilization. The OPD stock solution was stored at 4° C. 36 μl 30% H₂O₂ was added to 21 ml OPD stock solution right before each detection. The experiments included positive controls, negative control 1 and negative control 2. The positive controls were plates coated with M13 phage and detected with HRP-conjugated anti-M13 antibodies. The negative control 1 did not have biotinylated ERα36, and the negative control 2 did not have phage.

Following the above procedures, 40 positive phage clones expressing human anti-ERα36 scFv were identified from the phage single clones selected from the fourth round of the panning process. The results are listed in Table 5.

TABLE 5
ELISA results of the phage single clones

| Clone | ELISA Signal (OD490) | | |
|---|---|---|---|
| No. | Well 1 | Well 2 | Well 3 |
| 1 | 0.504 | 0.505 | 0.508 |
| 2 | 0.403 | 0.401 | 0.407 |
| 3 | 0.334 | 0.336 | 0.339 |
| 4 | 0.517 | 0.511 | 0.515 |
| 5 | 0.398 | 0.395 | 0.393 |
| 6 | 0.413 | 0.414 | 0.418 |
| 7 | 0.464 | 0.468 | 0.464 |
| 8 | 0.382 | 0.385 | 0.388 |
| 9 | 0.435 | 0.438 | 0.437 |
| 10 | 0.388 | 0.385 | 0.381 |
| 11 | 0.400 | 0.405 | 0.407 |
| 12 | 0.381 | 0.388 | 0.383 |
| 13 | 0.408 | 0.409 | 0.403 |
| 14 | 0.500 | 0.502 | 0.504 |
| 15 | 0.424 | 0.428 | 0.427 |
| 16 | 0.371 | 0.377 | 0.373 |
| 17 | 0.521 | 0.523 | 0.528 |
| 18 | 0.189 | 0.183 | 0.181 |
| 19 | 0.373 | 0.379 | 0.372 |
| 20 | 0.418 | 0.411 | 0.415 |
| 21 | 0.371 | 0.377 | 0.379 |
| 22 | 0.271 | 0.278 | 0.276 |
| 23 | 0.329 | 0.324 | 0.323 |
| 24 | 0.388 | 0.386 | 0.383 |
| 25 | 0.298 | 0.293 | 0.299 |
| 26 | 0.243 | 0.244 | 0.246 |
| 27 | 0.414 | 0.417 | 0.418 |
| 28 | 0.390 | 0.394 | 0.399 |
| 29 | 0.421 | 0.424 | 0.427 |
| 30 | 0.356 | 0.358 | 0.353 |
| 31 | 0.410 | 0.415 | 0.416 |
| 32 | 0.388 | 0.382 | 0.380 |
| 33 | 0.405 | 0.409 | 0.403 |
| 34 | 0.521 | 0.522 | 0.518 |
| 35 | 0.374 | 0.369 | 0.371 |
| 36 | 0.420 | 0.411 | 0.417 |
| 37 | 0.556 | 0.552 | 0.547 |
| 38 | 0.485 | 0.478 | 0.479 |
| 39 | 0.364 | 0.365 | 0.361 |
| 40 | 0.325 | 0.330 | 0.328 |
| N1 | 0.094 | 0.098 | 0.097 |
| N2 | 0.088 | 0.084 | 0.087 |
| P | 0.870 | 0.877 | 0.875 |

Notes:
P is positive control;
N1 is negative control 1;
and N2 is negative control 2.

### Example 3

### Nucleotide Sequencing of Human Anti-ERα36 scFv Expressed by Positive Phage Clones

Each DNA of the 40 positive phage clones was sequenced respectively using the following sequencing primer:

5′-TGGAATTGTGAGCGGATAACAATT-3′,

5′-GTAAATGAATTTCTGTATGAGG-3′. The sequencing results were studied using sequence analyzing software Vector NTI (purchased from Invitrogen) to obtain 7 different nucleotide sequences (SEQ ID NO: 4, SEQ ID NO: 6, SEQ ID NO: 8, SEQ ID NO: 10, SEQ ID NO: 12, SEQ ID NO: 14, and SEQ ID NO: 16, see description) and 7 predicted amino acid sequences (SEQ ID NO: 3, SEQ ID NO: 5, SEQ ID NO: 7, SEQ ID NO: 9, SEQ ID NO: 11, SEQ ID NO: 13, and SEQ ID NO: 15, see description). The 7 human anti-ERα36 scFv were labeled as ScFv1, ScFv2, ScFv3, ScFv4, ScFv5, ScFv6 and ScFv7, whose sequences are described in the corresponding exemplary embodiments supra. The 40 sequenced phage clones were grouped according to the 7 predicted amino acid sequences as shown in Table 6.

TABLE 6
Grouping of the positive phage clones based on the amino acid sequences
of the expressed anti-ERα36 ScFv

| Antibody sequence No. | Phage clone No. |
|---|---|
| ScFv1 (SEQ ID NO: 3) | 2, 5, 6, 9, 13, 19, 20, 29, 31, 33, |
| | 36 |
| ScFv2 (SEQ ID NO: 5) | 1, 7, 11, 15, 17, 25, 28, 34, 38 |
| ScFv 3 (SEQ ID NO: 7) | 4, 8, 14, 22, 23, 37 |
| ScFv 4 (SEQ ID NO: 9) | 10, 16, 21, 32, 35 |
| ScFv 5 (SEQ ID NO: 11) | 3, 30, 40, 18 |
| ScFv 6 (SEQ ID NO: 13) | 27, 39, 26 |
| ScFv 7 (SEQ ID NO: 15) | 12 |
| ND | 24 |

Notes:
ND means not determined.

### Example 4

### Expression and Purification of Human Anti-ERα36 ScFv1-ScFv7

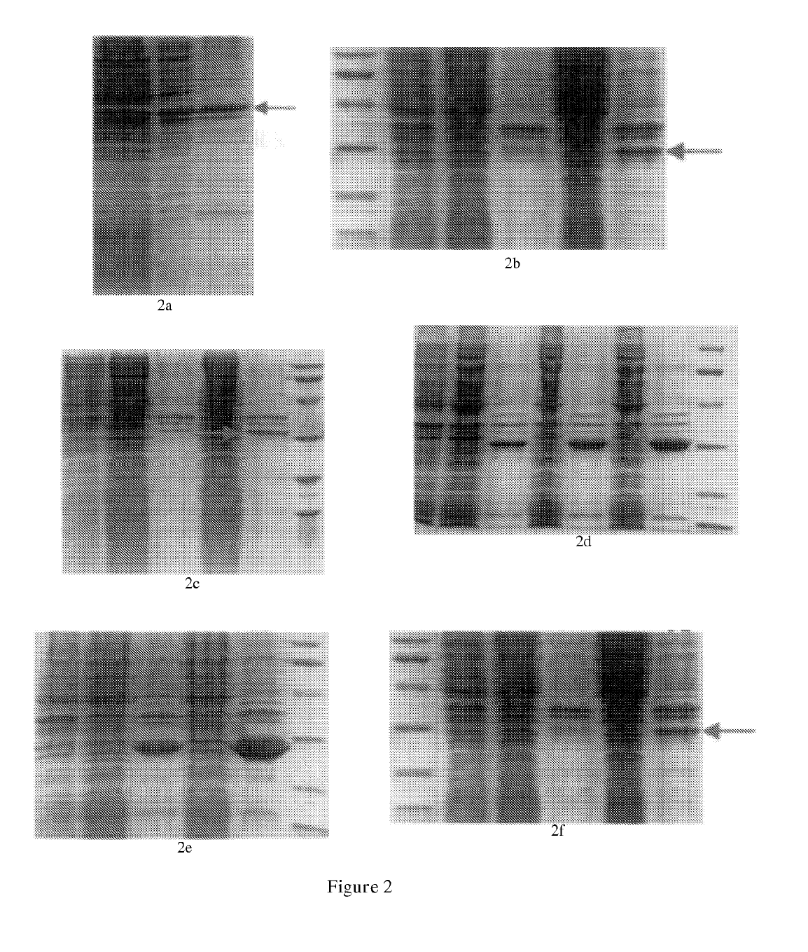

A single colony of HB2151 (Purchased from GE Healthcare) was picked from the LB plate and propagated at 37° C. in 2TY medium until OD600 reached 0.6-0.8. The phage clones (1 μl) identified in Example 3 which expressed ScFv1-ScFv7 were inoculated into 200 μl log-phase HB2151 culture respectively, and incubated for 30 min at 37° C. The infected cultures were plated onto LB-AG plates (LB medium containing Ampicillin and glucose) respectively and cultured at 37° C. overnight. 250 μl of the cultured cells were inoculated into 2TY medium (25 mL) for propagation at 37° C. until OD600 reached about 0.6. 250 μl 2TY induction medium (2TY containing 0.1 mM IPTG induction agent) was added to each culture, followed by overnight incubation at 30° C. Cells were collected by centrifugation at 3500 g for 10 min and suspended in PBS (1:50 v:v). The cell suspensions were treated with ultra-sonication to release soluble ScFv and centrifuged at 9000 g for 10 min to collect the supernatants containing the soluble ScFv. Supernatants containing ScFv1, ScFv2, ScFv3, ScFv4, ScFv5, ScFv6 and ScFv7 were labeled as S14, S24, S33, S41, S53, S66 and S72 respectively. Sodium dodecyl sulfate polyacrylamide gel electrophoresis (SDS-PAGE) was used to test the presence of the 7 ScFvs in the supernatants. The 7 ScFvs were found mainly in the supernatants, and they were all expressed in the form of inclusion bodies. FIG. 2 shows exemplary electrophoresis images of S14, S24, S33, S41, S66 and S72.

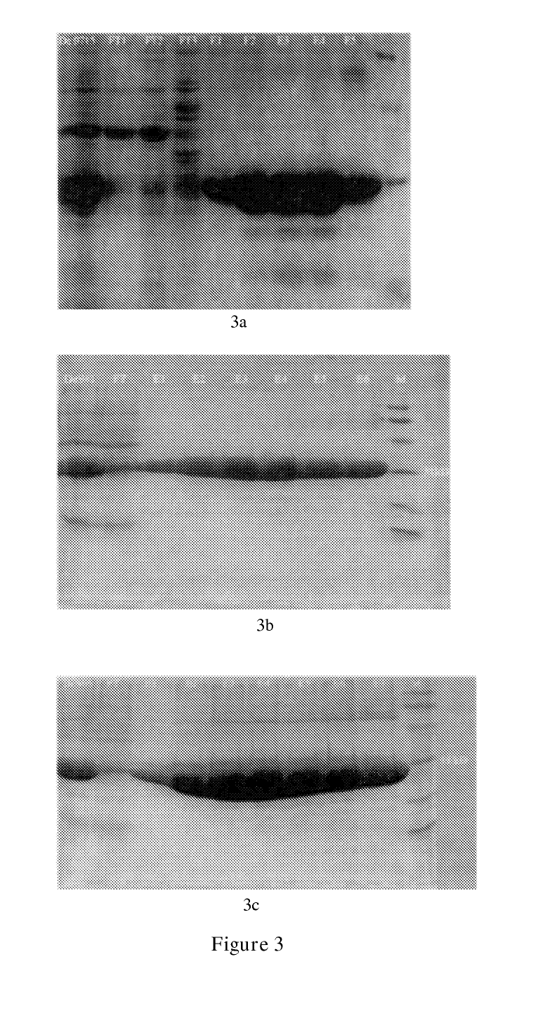

The 7 ScFvs were purified using Ni affinity chromatography under denaturing conditions because the ScFvs were expressed in inclusion bodies. The supernatants S14, S24, S33, S41, S53, S66 and S72 were denatured under the condition of 20 mM Tris.HCl, pH 8.0, 8 M urea and 50 mM beta-ME and then purified by Ni affinity chromatography. The 7 denatured inclusion body solutions were loaded onto the Ni affinity column (purchased from GE Healthcare). The column was washed with washing buffer (20 nM mM Tris.Cl, pH 8.0, 8 M urea, 10 mM imidaazole, 25 mM NaCl), and then equilibrated with equilibrium buffer (the same as the washing buffer) until the baseline was steady. The samples were eluted by gradient elution using elution buffer (equilibrium buffer with gradiently increased imidazole). The eluates were collected and analyzed using SDS-PAGE. FIG. 3a-3c shows the exemplary electrophoresis images of S14, S41 and S66 elutions respectively. The concentrated target bands and few impurity bands show high purity and effective purifications of the proteins in the eluates.

The eluates from Ni column chromatography were dialyzed in a ratio of 1:10 into dialysis buffer (50 mM borate saline buffer containing 8M urea, pH 8.9). The dialysis buffer was changed after 17 h of dialysis and the eluates were dialyzed for another 7 h. The dialyzed samples were diluted to 300 μg/ml using borate saline buffer (50 mM, pH 8.9, containing 8M urea), and re-natured in the ratio of 1:10 by dialysis. Half of the dialysis buffer was changed in each step of the renaturation dialysis. The compositions of the dialysis buffers used in each step were as follows:

50 mM borate saline buffer, 1% glycine, 4M urea, pH8.9;

50 mM borate saline buffer, 1% glycine, 2M urea, pH8.9;

50 mM borate saline buffer, 1% glycine, 1M urea, pH8.9;

50 mM borate saline buffer, 1% glycine, 0.5M urea, pH8.9;

50 mM borate saline buffer, 1% glycine, pH8.7;

50 mM borate saline buffer, 1% glycine, pH8.7;

50 mM borate saline buffer, 1% glycine, pH8.7.

0.4 M L-arginine was used in replacement of the 1% glycine for renaturation of some S24 samples.

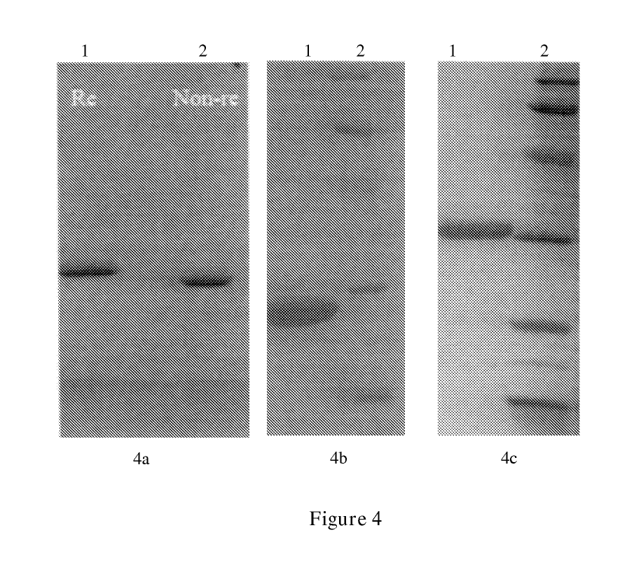

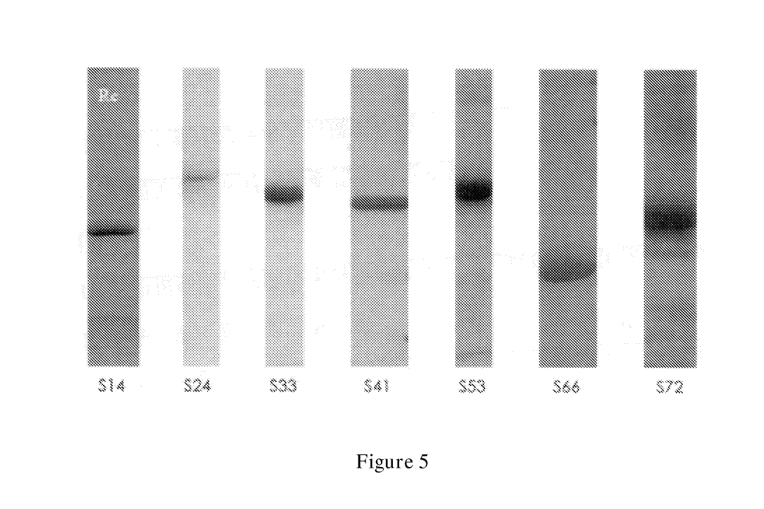

Several re-natured ScFv samples were obtained after renaturation of the supernatants containing the seven inclusion bodies. Table 7 lists the batch numbers, concentrations and total protein amounts of these re-natured samples. The results show that, most of the re-natured samples have high concentration of proteins. Almost all the samples have a total protein yield of above 10 mg, which may be used in future studies. The re-natured samples were analyzed using reduced SDS-PAGE. The exemplary electrophoresis images of the re-natured samples obtained from S14, S24, S33, S41, S53, S66 and S72 in reduced SDS-PAGE were shown in FIG. 4a-c and FIG. 5. In FIG. 4a-c, the positions of the target bands of S14, S41 and S66 do not show obvious changes before and after the renaturation, suggesting that the renaturation process did not significantly affect the molecular weights of the target proteins. FIG. 5 shows that only one target band was observed for each of the seven ScFv re-natured samples by SDS-PAGE, suggesting that the re-natured proteins had high purity. The 7 re-natured ScFv solutions were lyophilized in the presence of 0.15 M trehalose, and stored for future use.

TABLE 7
Yields of re-natured human anti-ERα36 ScFv1-ScFv7

| | | | | | Total |
|---|---|---|---|---|---|
| | | Total | Protein | | Protein |
| Batch | Concentration | volume | quantity | Corresponding | quantity |
| No. | (μg/ml) | (ml) | (mg) | ScFv No. | (mg) |
| S14A1r1 | 198.6 | 148 | 29.4 | ScFv1 | 29.4 |
| S24A1r1 | 13.89 | 152 | 2.1 | ScFv2 | 6 |
| S24A2r2 | 42.54 | 92 | 3.9 | | |
| S33A1r1 | 13.8 | 72 | 1 | ScFv3 | 12 |
| S33A2r1 | 167.9 | 56 | 9.4 | | |
| S33A3r1 | 23 | 72 | 1.6 | | |
| S41A1r1 | 80.8 | 44 | 3.6 | ScFv4 | 24.7 |
| S41A2r2 | 87.5 | 84 | 7.4 | | |
| S41A3r3 | 171.7 | 80 | 13.7 | | |
| S53A1r1 | 232 | 112 | 25.9 | ScFv5 | 25.9 |
| S66A1r1 | 234.8 | 148 | 34.8 | ScFv6 | 38.8 |
| S66A1r2 | 100 | 40 | 4 | | |
| S72A1r1 | 200 | 28 | 5.6 | ScFv7 | 18.9 |
| S72A2r1 | 242 | 28 | 6.7 | | |
| S72A3r1 | 97.9 | 68 | 6.6 | | |

Characterization of Human Anti-ERα36 ScFv1˜ScFv7 by Western Blot.

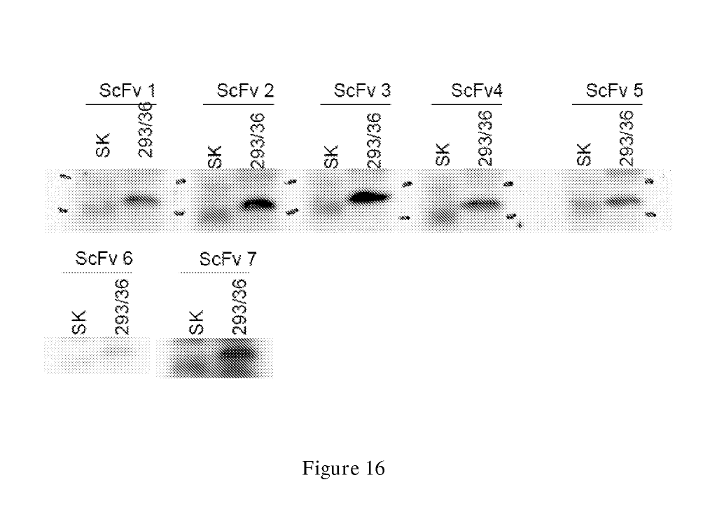

ScFv1˜ScFv7 obtained after purification and renaturation were characterized using Western Blot. HEK293 cells were artificially constructed to express recombinant ERα36. Human breast cancer cells SK-BR-3 and ERα36-expressing HEK293 cells were cultured, harvested, and lysed by lysis buffer to obtain the cell lysates respectively. 20 μg/lane of the lysates were then loaded for SDS-PAGE. After electrophoresis, the protein samples were electronically transferred to PVDF membrane labeled with ScFv1˜ScFv7 respectively. The membranes were blocked with non-fat milk and anti-His-HRP was added to show the resulting images (FIG. 16).

The Western Blot results show that ScFv1˜ScFv7 can be used as antibodies to specifically detect the presence of recombinant ERα36, suggesting that ScFv1˜ScFv7 were capable of specific binding with ERα36.

### Example 5

### Characterization of Binding Affinity of Human Anti-ERα36 ScFv1˜ScFv7

ELISA of ScFv.

Streptavidin-coated 96-well plates were washed twice with PBS. Biotinylated ERα36 (final concentration of 20 μg/ml) was added into each testing well. The plates were incubated for 2 h at room temperature and washed three times with PBST. Re-natured ScFv samples (100 μl, S14A1r1, S24A2r2, S33A2r1, S41A3r3, S53A1r1, S66A1r1 and S72A2r1) were added into the testing wells respectively. Each ScFv was diluted five times in five gradients, and two parallel wells were loaded with each ScFv at each gradient. The plates were incubated at 37° C. for 1 h. The plates were washed three times with PBST, followed by addition of 100 μl anti-His-6 mouse monoclonal antibody (1:2000 dilution) per well and 1 h incubation at 37° C. The plates were washed three times with PBST, followed by addition of 100 μl goat anti-mouse-HRP (1:2500 dilution) per well and 1 h incubation at 37° C. The plates were washed six times with PBST, followed by addition of 100 μl OPD to develop under the protection from light. The reactions were terminated by adding 50 μl 2M H₂SO₄ to each well, and data were collected by microplate reader at 490 nm.

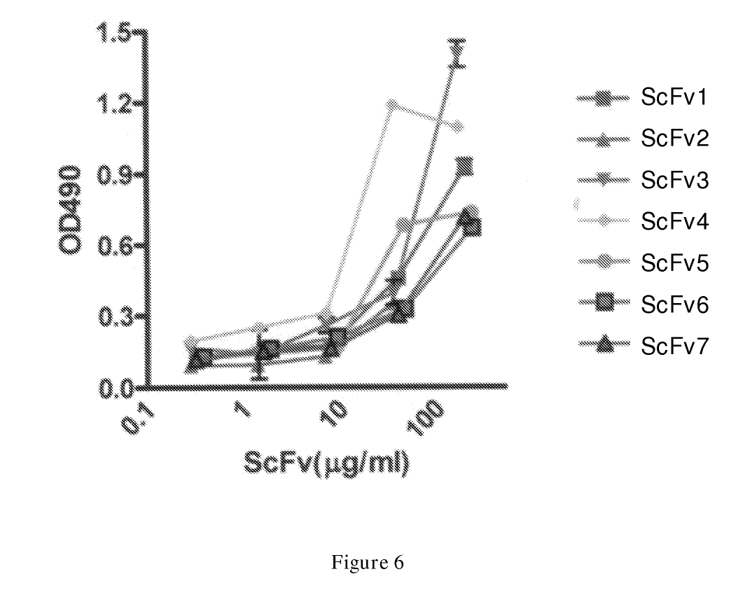

One positive control and three negative controls were performed in parallel. The positive control used rabbit anti-ER multi-clonal antibody serum in replacement of the ScFv re-natured sample and goat-anti-rabbit-HRP in replacement of goat-anti-mouse HRP. Negative control 1 (N1) did not contain ScFv re-natured sample. Negative control 2 (N2) used 200 μg/ml ScFv re-natured sample, but did not contain His-6 mono-clonal antibody. Negative control 3 (N3) did not contain either ScFv re-natured sample or His-6 mono-clonal antibody. The experimental results were shown in Table 8. The OD490 values were plotted against the concentrations of the 7 ScFvs, and were shown in FIG. 6. ScFv1-ScFv7 demonstrated notable concentration dependent binding affinity to ERα36 at the tested concentrations, suggesting that ScFv1-ScFv7 can specifically bind with ERα36.

TABLE 8
ELISA data of human anti-ERα36 ScFv1-ScFv7 binding to ERα36

| | |
|---|---|
| ScFv | OD490 |

| | concentration (μg/ml) | Well1 | Well2 |
|---|---|---|---|
| ScFv1 | 200.00 | 0.959 | 0.898 |
| ScFv1 | 40.00 | 0.461 | 0.456 |
| | 8.00 | 0.177 | 0.157 |
| | 1.60 | 0.165 | 0.157 |
| | 0.32 | 0.128 | 0.122 |
| ScFv2 | 35.00 | 0.343 | 0.359 |
| | 7.00 | 0.138 | 0.121 |
| | 1.40 | 0.095 | 0.096 |
| | 0.28 | 0.093 | 0.090 |
| ScFv3 | 170.00 | 1.459 | 1.349 |
| | 36.00 | 0.448 | 0.344 |
| | 7.20 | 0.290 | 0.229 |
| | 1.44 | 0.242 | 0.037 |
| | 0.28 | 0.159 | 0.171 |
| ScFv4 | 170.00 | 1.087 | 1.103 |
| | 36.00 | 1.177 | 1.200 |
| | 7.20 | 0.299 | 0.320 |
| | 1.44 | 0.254 | 0.248 |
| | 0.28 | 0.205 | 0.187 |
| ScFv5 | 232.00 | 0.743 | 0.725 |
| | 46.00 | 0.676 | 0.687 |
| | 9.20 | 0.168 | 0.169 |
| | 1.80 | 0.148 | 0.137 |
| | 0.36 | 0.124 | 0.128 |
| ScFv6 | 235.00 | 0.677 | 0.664 |
| | 47.00 | 0.327 | 0.326 |
| | 9.40 | 0.210 | 0.209 |
| | 1.90 | 0.161 | 0.162 |
| | 0.38 | 0.127 | 0.129 |
| ScFv7 | 200.00 | 0.690 | 0.740 |
| | 40.00 | 0.294 | 0.311 |
| | 8.00 | 0.171 | 0.155 |
| | 1.60 | 0.146 | 0.155 |
| | 0.32 | 0.122 | 0.111 |
| P | 2.287 | 2.311 | |
| N1 | 0.081 | 0.080 | |
| N2 | 0.323 | 0.336 | |
| N3 | 0.062 | 0.083 | |

Note:
P is positive control,
N1 is negative control 1,
N2 is negative control 2 and
N3 is negative control 3.

### Example 6

### Tumor Growth Inhibition Studies of Human Anti-ERα36 ScFv1, ScFv3, ScFv4, ScFv7 and Rabbit Anti-ERα36 Multi-Clonal Antibody on Tumor Bearing Mice

Animals.

49 female BALB/c-nu nude mice (provided by department of laboratory animal science, Peking University Health Science Center) were used in the studies.

Grouping.

The tested animals were grouped into 7 groups with 7 nude mice in each group. The seven groups are the negative control group treated with human IgG antibody (purchased from Beijing Biosynthesis Biotechnology Co. Ltd.), the positive control group treated with herceptin (provided by Beijing Shenogen pharma group), the multi-clonal antibody test group treated with rabbit anti-ERα36 multi-clonal antibody (provided by Wang, Zhao-Yi group, Creighton University), and the monoclonal antibody test groups treated with human anti-ERα36 monoclonal ScFv1, ScFv3, ScFv4 and ScFv7 respectively.

Drug Administration.

The test groups were administered with the same dose of rabbit anti-ERα36 multi-clonal antibody, human anti-ERα36 monoclonal ScFv1, ScFv3, ScFv4 and ScFv7 respectively (5 mg/kg, 100 μg/dose). The positive control group was administered with 5 mg/kg herceptin (100 μg/dose,), and the negative control group was administered with 5 mg/kg IgG antibody (100 μg/dose).

Methods and Data Analysis.

Human breast tumor BCAP-37 (estrogen receptor positive, provided by department of pharmacology, Institute of Materia Medica, Chinese Academy of Medical Sciences) was implanted subcutaneously into the armpit of the right forelimb of each tested nude mouse. Diethylstilbestrol was administered by gavage with the dose of 7 μg/day for 9 consecutive days after the implantation. On day 10, the mice were grouped into 7 groups and administered with the corresponding drug via intravenous injection, at a dosing interval of 3-4 days, for 6 consecutive doses. Every 3-4 days, the tumor-bearing mice were weighed and their tumor sizes were measured. Parameters were calculated using the following equations:

Volume of Tumor in animals of each group (VT): Vt=½×a×b², (Equation 1),

wherein a and b represent the length and width of the tumor, respectively;

$$Relative Volume of Tumor ( R V T ) \text{:} R T V = \frac{{V}_{t}}{{V}_{0}} , \qquad ( Equation 2 ) ,$$

wherein V₀ is the volume of tumor before drug administration at the time of grouping, Vₜ is the volume of tumor at each measurement; the calculation method of V₀ is the same as that of Vₜ;

$$Tumor Growth Rate T \text{/} C ( \% ) \text{:} TC \% = \frac{T R T V}{C R T V } \times 100 \% , \qquad ( Equation 3 ) ,$$

wherein TRTV is the arithmetic mean of RTV of the test groups, and CRTV is the arithmetic mean of RTV of the negative control group.

The tumor bearing mice in each group were terminated after 21 days of observations and drug administrations. The tumors were then excised from the animals, photographed and weighed on 1/10000 analytical balance. The average tumor weights and tumor growth inhibition rates were calculated:

$$TumorGrowthInhibitionRate = \frac{{AverageTumorWeight}_{NegativeControl} ( g ) - {AverageTumorWeight}_{TreatedGroup} ( g )}{{AverageTumorWeight}_{NegativeControl} ( g )} . \qquad ( Equation 4 )$$

All experimental data were shown in mean±standard deviation. Groups were compared using statistical t test.

Results.

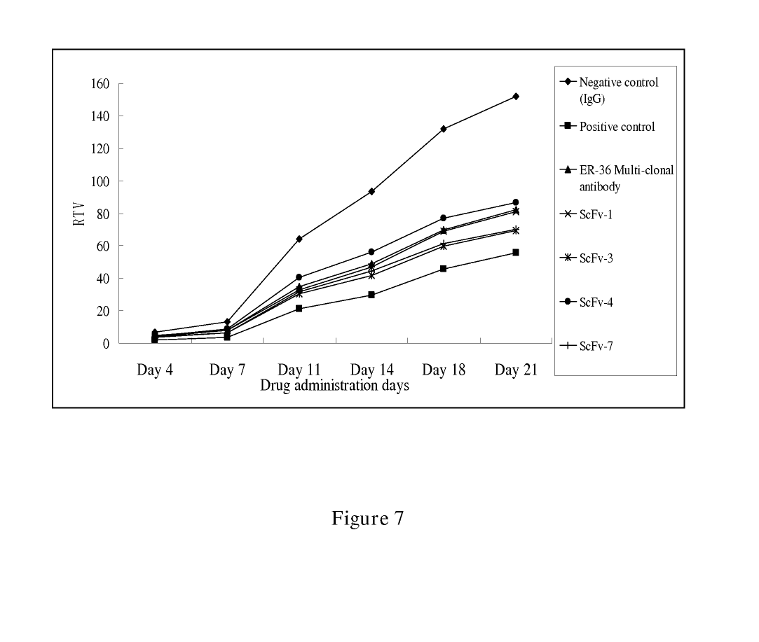

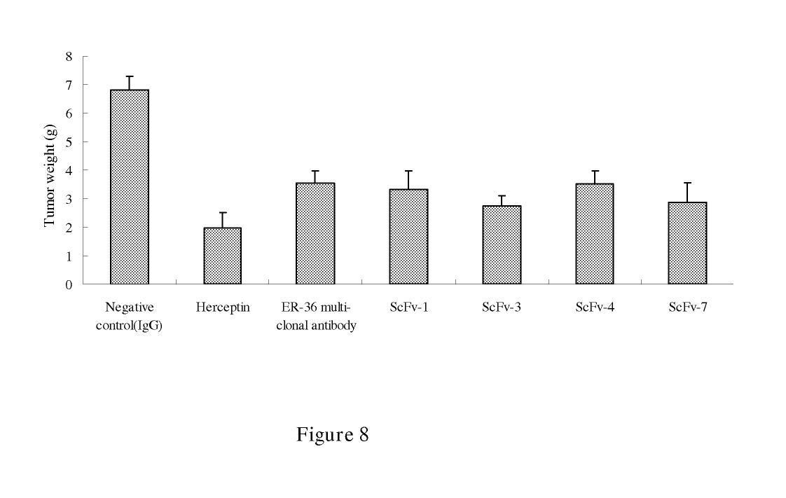

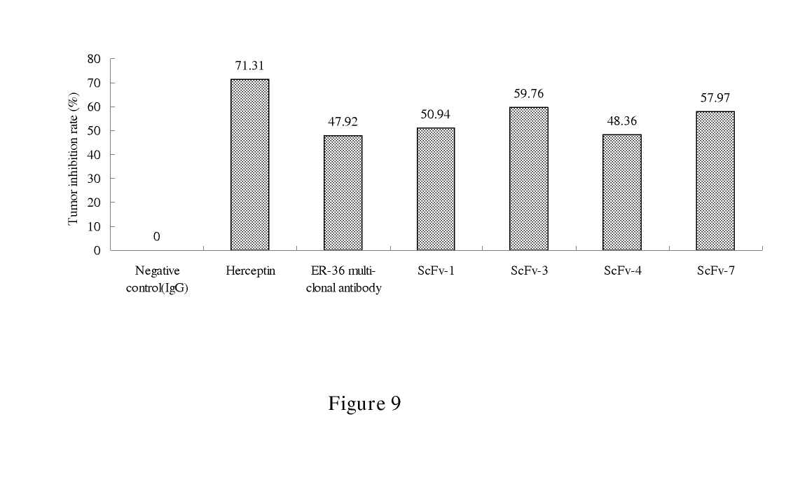

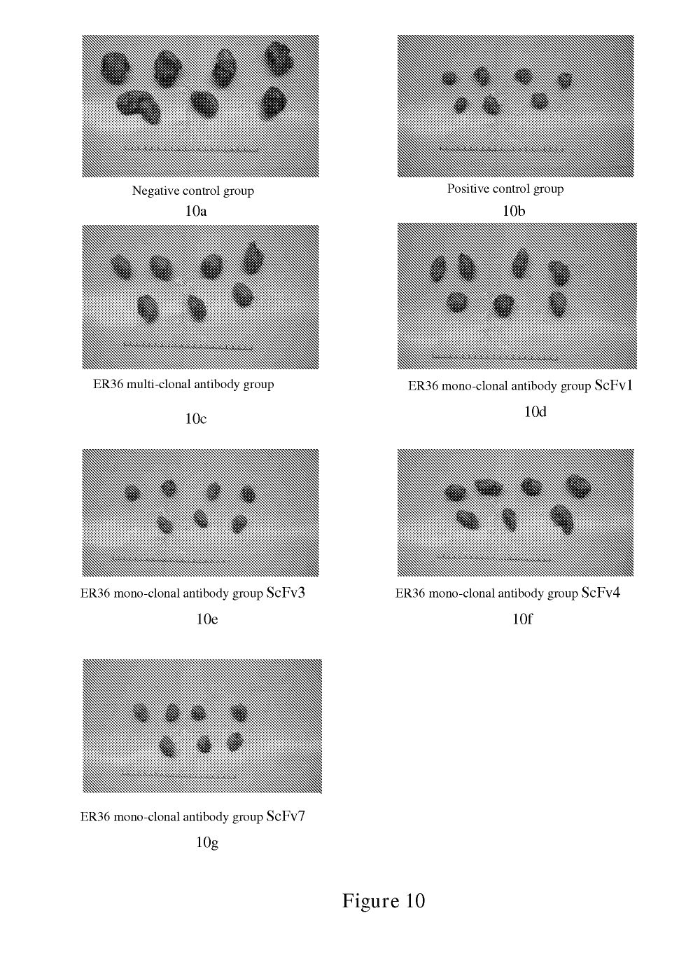

All five test antibodies were dissolved well in sterilized water to form clear solutions. No tumor-bearing mice died during the drug administration period. The body weights of the mice did not show significant differences between the test groups and the negative control groups (see Table 9 and Table 10). According to the observed tumor volume (TV) (see Table 11 and Table 12), relative tumor volume (RTV) (see Table 13, Table 14 and FIG. 7), relative tumor growth rate (T/C), actual tumor weight (see Table 15 and FIG. 8) and tumor growth inhibition rate (see Table 16 and FIG. 9) of the tumor bearing mice, all of the five test antibodies significantly inhibited tumor growth in the tumor-bearing mice, wherein the ScFv-3 group and the ScFv-7 group showed the strongest inhibitory activities. The inhibitory effects on tumor growth of the five test antibodies were slightly less than that of the positive control herceptin group. Pathological examination of the 7 groups (see FIG. 10) showed that, the tumor sizes in the negative control group were significantly larger than those of the positive control group, as well as those of all the test groups. In the test groups, the tumor sizes were slightly larger in the multi-clonal antibody group than those in the mono-clonal antibody groups, but they are still significantly smaller than those in the negative control group. The tumors of ScFv-3 group and ScFv-7 group were the smallest among the mono-clonal antibody groups, suggesting that ScFv-3 and ScFv-7 had an anti-tumor effect close to that of the positive control herceptin. The tumor sizes of ScFv-1 group and ScFv-4 group were significantly smaller when compared with those of the negative control group, and were comparable to those of the multi-clonal antibody group. But they were slightly larger than those of the positive control group.

TABLE 9
Grouping of the tumor-bearing mice, doses and body weights at the time
of grouping

| | Number of | body weight | |
|---|---|---|---|
| | animals in the | when | |
| Group | group | grouped | Dose |
| Negative control group | 7 | 19.82 ± 0.44 | 5 mg/kg |
| Positive control group | 7 | 19.87 ± 0.44 | 5 mg/kg |
| Multi-clonal antibody | 7 | 19.88 ± 0.57 | 5 mg/kg |
| group | | | |
| Mono-clonal antibody | 7 | 19.91 ± 0.49 | 5 mg/kg |
| ScFv-1 group | | | |
| Mono-clonal antibody | 7 | 19.94 ± 0.81 | 5 mg/kg |
| ScFv-3 group | | | |
| Mono-clonal antibody | 7 | 19.67 ± 0.57 | 5 mg/kg |
| ScFv-4 group | | | |
| Mono-clonal antibody | 7 | 19.67 ± 0.67 | 5 mg/kg |
| ScFv-7 group | | | |

TABLE 10
Body weights of tumor-bearing nude mice during the drug administration period

| | | Day 4 | Day 7 | Day 11 | Day 14 | Day 18 | Day 21 |
|---|---|---|---|---|---|---|---|
| | Before drug | after drug | after drug | after drug | after drug | after drug | after drug |
| Group | administration | administration | administration | administration | administration | administration | administration |
| Negative | 19.82 ± 0.44 | 20.08 ± 0.42 | 20.45 ± 0.35 | 21.64 ± 0.63 | 22.21 ± 0.52 | 23.41 ± 0.49 | 23.88 ± 0.27 |
| control group | | | | | | | |
| Positive | 19.87 ± 0.44 | 20.27 ± 0.39 | 20.74 ± 0.33 | 21.34 ± 0.42 | 21.98 ± 0.41 | 22.97 ± 0.44 | 23.62 ± 0.46 |
| control group | | | | | | | |
| Multi-clonal | 19.88 ± 0.57 | 20.28 ± 0.39 | 20.65 ± 0.38 | 21.34 ± 0.42 | 22.01 ± 0.36 | 23.35 ± 0.65 | 23.70 ± 0.59 |
| antibody group | | | | | | | |
| ScFv-1 group | 19.91 ± 0.49 | 20.25 ± 0.40 | 20.74 ± 0.32 | 21.32 ± 0.41 | 22.05 ± 0.35 | 23.62 ± 0.43 | 23.84 ± 0.41 |
| ScFv-3 group | 19.94 ± 0.81 | 20.28 ± 0.54 | 20.75 ± 0.31 | 21.24 ± 0.55 | 22.04 ± 0.59 | 23.38 ± 0.46 | 23.62 ± 0.36 |
| ScFv-4 group | 19.67 ± 0.57 | 20.07 ± 0.48 | 20.72 ± 0.28 | 21.44 ± 0.51 | 22.01 ± 0.46 | 23.47 ± 0.39 | 23.64 ± 0.25 |
| ScFv-7 group | 19.67 ± 0.67 | 20.02 ± 0.51 | 20.65 ± 0.24 | 21.34 ± 0.51 | 22.01 ± 0.52 | 23.15 ± 0.58 | 23.52 ± 0.47 |

Note:
No significant difference observed when compared with negative control group.

TABLE 11
Tumor volumes (mm³) of the tumor-bearing nude
mice during the drug administration period

| | | Day 4 | Day 7 | Day 11 |
|---|---|---|---|---|
| | Before drug | after drug | after drug | after drug |
| Group | administration | administration | administration | administration |
| Negative | 50.48 ± 11.72 | 350.70 ± 85.00 | 663.67 ± 196.02 | 3159.09 ± 593.30^{b} |
| control group | | | | |
| Positive | 50.95 ± 15.31 | 89.08 ± 25.89^{b} | 186.80 ± 58.36^{b} | 1049.69 ± 251.43^{b} |
| control group | | | | |
| Multi-clonal | 50.45 ± 12.48 | 228.78 ± 44.33^{b} | 387.25 ± 66.52^{b} | 1693.71 ± 389.00^{b} |
| antibody group | | | | |
| Mono-clonal | 50.83 ± 16.51 | 184.31 ± 81.10^{b} | 344.75 ± 135.70^{b} | 1493.66 ± 517.68^{b} |
| antibody | | | | |
| ScFv-1 group | | | | |
| Mono-clonal | 50.51 ± 10.12 | 171.91 ± 73.68^{b} | 302.93 ± 100.01^{b} | 1442.70 ± 406.32^{b} |
| antibody | | | | |
| ScFv-3 group | | | | |
| Mono-clonal | 50.25 ± 13.96 | 222.27 ± 104.89^{b} | 427.50 ± 136.98^{a} | 1930.04 ± 390.85^{b} |
| antibody | | | | |
| ScFv-4 group | | | | |
| Mono-clonal | 50.62 ± 13.32 | 186.11 ± 71.13^{a} | 308.85 ± 117.52^{b} | 1521.45 ± 366.24^{b} |
| antibody | | | | |
| ScFv-7 group | | | | |

| | Day 14 | Day 18 | Day 21 |
|---|---|---|---|
| | after drug | after drug | after drug |
| Group | administration | administration | administration |
| Negative | 4501.79 ± 530.09 | 6281.90 ± 732.53 | 7287.01 ± 889.55^{b} |
| control group | | | |
| Positive | 1394.53 ± 201.24^{b} | 2141.61 ± 276.39^{b} | 2586.37 ± 233.62^{b} |
| control group | | | |
| Multi-clonal | 2346.03 ± 506.84^{b} | 3317.76 ± 713.86^{b} | 3919.27 ± 642.95^{b} |
| antibody group | | | |
| Mono-clonal | 2174.87 ± 514.19^{b} | 3208.12 ± 595.60^{b} | 3792.10 ± 639.19^{b} |
| antibody | | | |
| ScFv-1 group | | | |
| Mono-clonal | 1993.02 ± 405.84^{b} | 2859.65 ± 427.16^{b} | 3324.97 ± 460.89^{b} |
| antibody | | | |
| ScFv-3 group | | | |
| Mono-clonal | 2666.60 ± 464.32^{b} | 3644.40 ± 414.64^{b} | 4098.31 ± 557.11^{b} |
| antibody | | | |
| ScFv-4 group | | | |
| Mono-clonal | 2136.49 ± 479.95^{b} | 2944.61 ± 526.75^{b} | 3358.43 ± 445.13^{b} |
| antibody | | | |
| ScFv-7 group | | | |

Note:
^{a}p < 0.05 when comparing with negative control;
^{b}p < 0.01 when comparing with negative control.

TABLE 12
Statistical comparisons (P value) of tumor volumes (mm³) between different groups

| Statistical | Positive | Multi-clonal | ScFv-1 | ScFv-3 | ScFv-4 | ScFv-7 |
|---|---|---|---|---|---|---|
| comparison | control | antibody group | group | group | group | group |
| Compared with | 1.3E−08 | 3.2E−06 | 2.2E−06 | 2.2E−07 | 3.6E−06 | 2.2E−07 |
| negative control | | | | | | |
| group | | | | | | |
| Compared with | — | 0.00024 | 0.00053 | 0.00261 | 2.5E−05 | 0.00157 |
| positive control | | | | | | |
| group | | | | | | |
| Compared with | — | — | 0.71702 | 0.07016 | 0.5879 | 0.08208 |
| multi-clonal | | | | | | |
| antibody group | | | | | | |
| Compared with | — | — | — | 0.1427 | 0.35819 | 0.16648 |
| ScFv-1 group | | | | | | |
| Compared with | — | — | — | — | 0.01518 | 0.89242 |
| ScFv-3 group | | | | | | |
| Compared with | — | — | — | — | — | 0.01776 |
| ScFv-4 group | | | | | | |

TABLE 13
Variations in relative tumor volume (RTV) of each group during the drug administration period

| Group | Day 4 | Day 7 | Day 11 | Day 14 | Day 18 | Day 21 |
|---|---|---|---|---|---|---|
| Negative | 6.98 ± 1.12 | 13.30 ± 3.47 | 64.00 ± 10.50 | 93.40 ± 23.10 | 132.00 ± 37.50 | 1520 ± 40.50^{b} |
| control group | | | | | | |
| Positive | 1.83 ± 0.59^{b} | 3.77 ± 0.89^{b} | 21.08 ± 2.54^{b} | 29.50 ± 8.83^{b} | 45.73 ± 15.18^{b} | 55.62 ± 19.51^{b} |
| control group | | | | | | |
| Multi-clonal | 4.77 ± 1.36^{b} | 8.02 ± 2.06^{b} | 35.00 ± 9.37^{b} | 48.86 ± 14.15^{b} | 69.72 ± 23.63^{b} | 82.17 ± 24.69^{b} |
| antibody group | | | | | | |
| Mono-clonal | 3.93 ± 2.06^{b} | 7.96 ± 4.25^{a} | 32.81 ± 15.25^{b} | 46.89 ± 17.08^{b} | 68.84 ± 22.84^{b} | 81.12 ± 25.35^{b} |
| antibody | | | | | | |
| ScFv-1 group | | | | | | |
| Mono-clonal | 3.61 ± 2.03^{b} | 6.31 ± 2.88^{b} | 30.44 ± 14.49^{b} | 41.87 ± 16.82^{b} | 59.61 ± 20.46^{b} | 69.18 ± 22.85^{b} |
| antibody | | | | | | |
| ScFv-3 group | | | | | | |
| Mono-clonal | 4.52 ± 2.15^{a} | 8.71 ± 2.75^{a} | 40.50 ± 10.88^{b} | 56.18 ± 14.99^{b} | 76.87 ± 19.31^{b} | 86.66 ± 23.90^{b} |
| antibody | | | | | | |
| ScFv-4 group | | | | | | |
| Mono-clonal | 3.81 ± 1.54^{b} | 6.45 ± 2.86^{b} | 31.64 ± 10.24^{b} | 44.48 ± 13.69^{b} | 61.54 ± 18.25^{b} | 70.27 ± 19.61^{b} |
| antibody | | | | | | |
| ScFv 7 group | | | | | | |

Note:
^{a}p < 0.05 when comparing with negative control;
^{b}denotes p < 0.01 when comparing with negative control.

TABLE 14
Statistical comparisons (P value) of tumor volumes (mm³) between
different groups after 21 days of drug administration

| Statistical | Positive | Multi-clonal | ScFv-1 | ScFv-3 | ScFv-4 | ScFv-7 |
|---|---|---|---|---|---|---|
| comparison | control | antibody group | group | group | group | group |
| Compared with | 1E−04 | 0.002 | 0.002 | 5E−04 | 0.003 | 4E−04 |
| negative control | | | | | | |
| group | | | | | | |
| Compared with | — | 0.045 | 0.057 | 0.255 | 0.021 | 0.186 |
| positive control | | | | | | |
| group | | | | | | |
| Compared with | — | — | 0.939 | 0.327 | 0.735 | 0.338 |
| multi-clonal | | | | | | |
| antibody group | | | | | | |
| Compared with | — | — | — | 0.373 | 0.681 | 0.388 |
| ScFv-1 group | | | | | | |
| Compared with | — | — | — | — | 0.187 | 0.925 |
| ScFv-3 group | | | | | | |
| Compared with | — | — | — | — | — | 0.186 |
| ScFv-4 group | | | | | | |

TABLE 15
Statistical comparisons (P value) of tumor volumes (mm³) between
different groups after 21 days of drug administration

| Statistical | Positive | Multi-clonal | ScFv-1 | ScFv-3 | ScFv-4 | ScFv-7 |
|---|---|---|---|---|---|---|
| comparison | control | antibody group | group | group | group | group |
| Compared with | 8.759E−10 | 1.641E−08 | 7.685E−08 | 4.645E−10 | 2.113E−08 | 3.713E−08 |
| negative control | | | | | | |
| groups | | | | | | |
| Compared with | — | 7.789E−05 | 0.00102 | 0.00902 | 0.00011 | 0.0207 |
| positive control | | | | | | |
| groups | | | | | | |
| Compared with | — | — | 0.491 | 0.0023 | 0.902 | 0.047 |
| multi-clonal | | | | | | |
| antibody groups | | | | | | |
| Compared with | — | — | — | 0.047 | 0.564 | 0.202 |
| ScFv-1 groups | | | | | | |
| Compared with | — | — | — | — | 0.004 | 0.686 |
| ScFv-3 groups | | | | | | |
| Compared with | — | — | — | — | — | 0.061 |
| ScFv-4 groups | | | | | | |

TABLE 16
Comparisons of variation in the tumor weights (g) and tumor growth
inhibition rates (%)

| | | Tumor | p- | Tumor growth |
|---|---|---|---|---|
| Groups | Number | weight (g) | value | inhibition rate (%) |
| Negative | 7 | 6.805 ± 0.487 | — | — |
| control | | | | |
| group | | | | |
| Positive | 7 | 1.952 ± 0.572 | <0.001 | 71.31 |
| control | | | | |
| group | | | | |
| Multi-clonal | 7 | 3.544 ± 0.434 | <0.001 | 47.92 |
| antibody | | | | |
| group | | | | |
| ScFv-1 | 7 | 3.338 ± 0.631 | <0.001 | 50.94 |
| group | | | | |
| ScFv-3 | 7 | 2.738 ± 0.344 | <0.001 | 59.76 |
| group | | | | |
| ScFv-4 | 7 | 3.514 ± 0.465 | <0.001 | 48.36 |
| group | | | | |
| ScFv-7 | 7 | 2.860 ± 0.696 | <0.001 | 57.97 |
| group | | | | |

Note:
p-values are calculated using statistical comparison between each treated group and the negative control group.

### Example 7

### Comparison of Anti-Tumor Activity of Human Anti-ERα36 ScFv-7 with Tamoxifen on Tumor-Bearing Nude Mice

Animals and Grouping.

BALB/c-nu female nude mice were divided into 3 groups, with 7 mice in each group.

Dose.

Human anti-ERα36 ScFv-7 was administered at the dose of 0.1 mg/20 g body weight/day. The positive control tamoxifen was administered at the dose of 0.33 mg/20 g body weight/day. The negative control group was administered with human IgG.

Methods and Data Analysis.

The tumor-bearing nude mice were weighed, and the volumes of the implanted tumors were measured every 3-4 days. The relative tumor volumes and tumor growth inhibition rates were calculated using Equation 2 and Equation 4 respectively. Animals received drug administration for 18 days and were terminated 24 h later. The tumors were excised from the animals and weighed. The average tumor weights and tumor growth inhibitions were calculated using Equation 4.

Results.

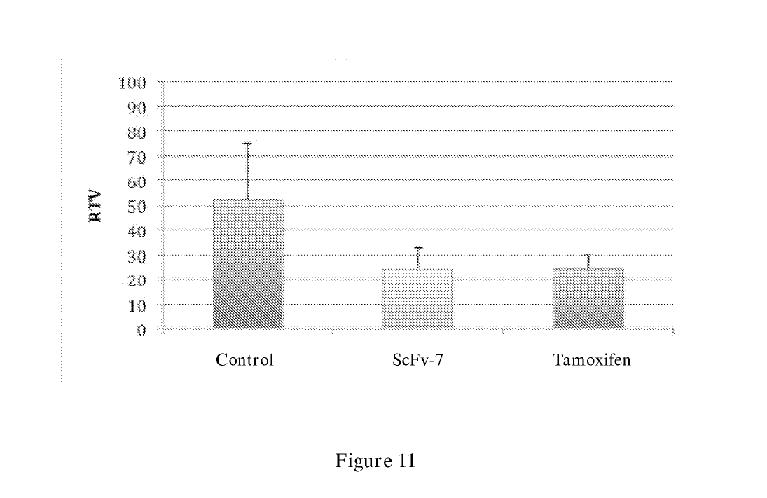

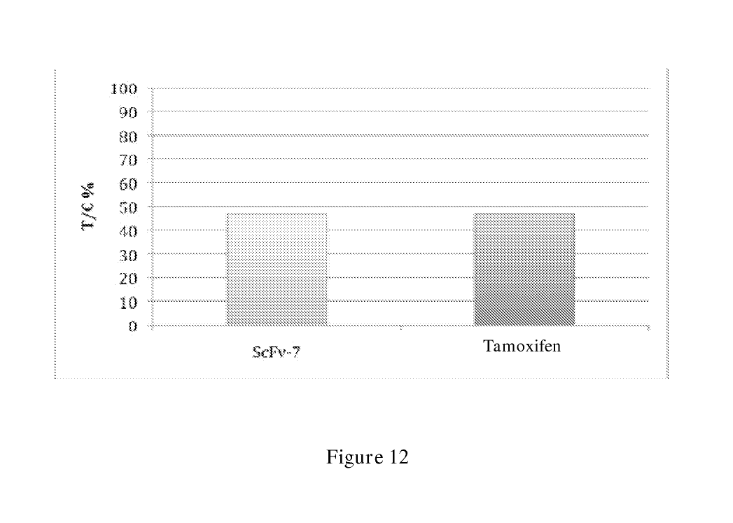

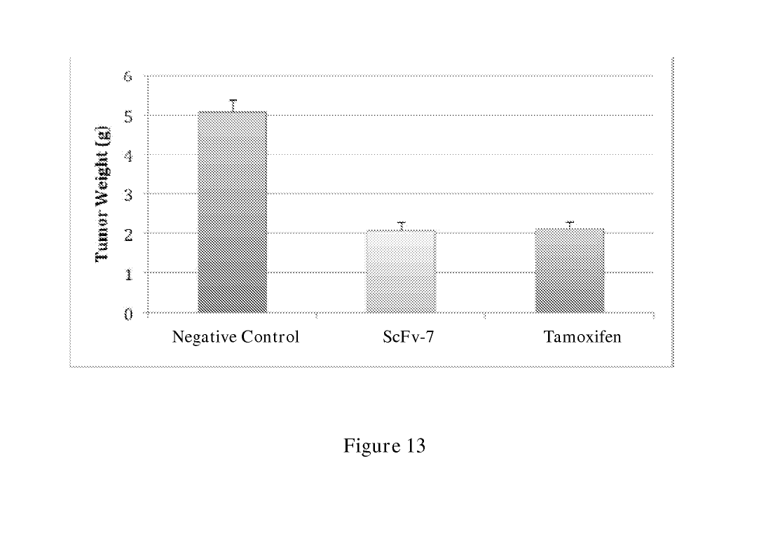

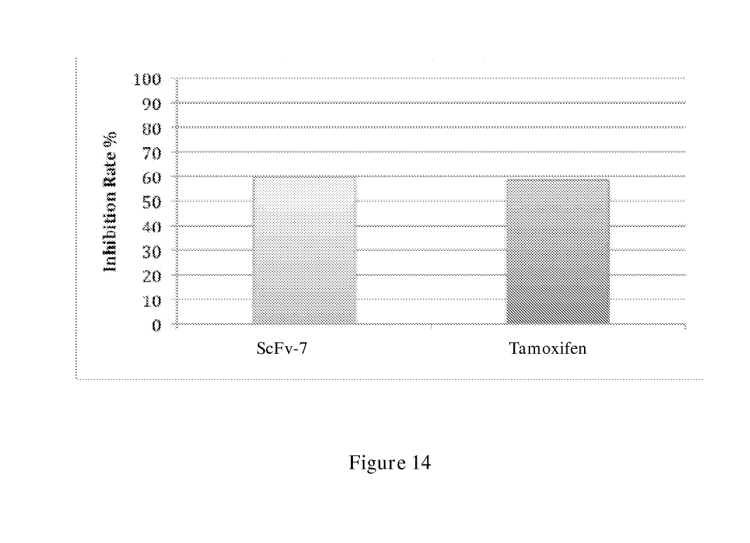

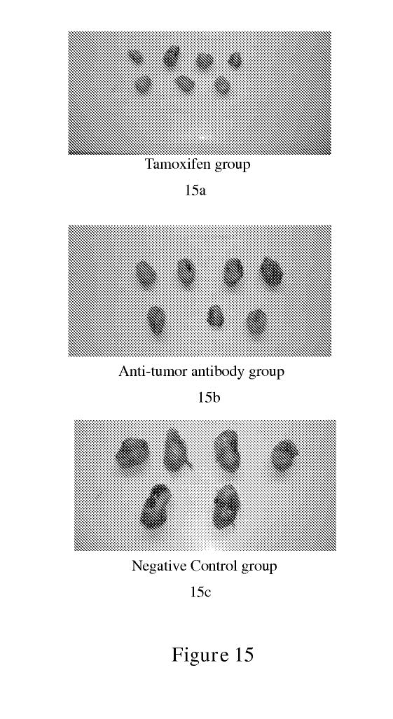

After 18 days of drug administration, the test group showed significantly lower levels of tumor volume (VT), relative tumor volume (RVT) (see FIG. 11) and relative tumor growth rate (T/C %) (see FIG. 12) than the parallel negative control group. The average tumor weights of the test group and the positive group showed significant difference when compared with those of the negative group, with p-values below 0.01 (see FIG. 13). The test group and the positive control group showed similar tumor inhibition rates of around 60% (see FIG. 14). The pathological examination results (see FIG. 15) of the three groups of tumor bearing mice showed that, the sizes of the tumors of the negative group were remarkably larger than those of the positive group. The tumor sizes of the test group were significantly smaller than those of the negative group, and were close to those of the positive control group treated with tamoxifen.

1. An antibody or antigen-binding fragment thereof which substantially binds to the same epitope to which ScFv 1 (SEQ ID NO 3), ScFv 2 (SEQ ID NO 5), ScFv 3 (SEQ ID NO 7), ScFv 4 (SEQ ID NO 9), ScFv 5 (SEQ ID NO 11), ScFv 6 (SEQ ID NO 13), or ScFv 7 (SEQ ID NO 15) specifically binds.

2. An antibody or antigen-binding fragment thereof comprising one or more members selected from the group consisting of ScFv 1 HCDR1, ScFv 2 HCDR1, ScFv 3 HCDR1, ScFv 4 HCDR1, ScFv 5 HCDR1, ScFv 6 HCDR1, ScFv 7 HCDR1, ScFv 1 HCDR2, ScFv 2 HCDR2, ScFv 3 HCDR2, ScFv 4 HCDR2, ScFv 5 HCDR2, ScFv 6 HCDR2, ScFv 7 HCDR2, ScFv 1 HCDR3, ScFv 2 HCDR3, ScFv 3 HCDR3, ScFv 4 HCDR3, ScFv 5 HCDR3, ScFv 6 HCDR3, ScFv 7 HCDR3, ScFv 1 LCDR1, ScFv 2 LCDR1, ScFv 3 LCDR1, ScFv 4 LCDR1, ScFv 5 LCDR1, ScFv 6 LCDR1, ScFv 7 LCDR1, ScFv 1 LCDR2, ScFv 2 LCDR2, ScFv 3 LCDR2, ScFv 4 LCDR2, ScFv 5 LCDR2, ScFv 6 LCDR2, ScFv 7 LCDR2, ScFv 1 LCDR3, ScFv 2 LCDR3, ScFv 3 LCDR3, ScFv 4 LCDR3, ScFv 5 LCDR3, ScFv 6 LCDR3, and ScFv 7 LCDR3.

3. The antibody or antigen-binding fragment thereof of claim 2, comprising one or more members selected from the group consisting of:
a) a heavy chain variable region comprising HCDR3, HCDR1, and/or HCDR2 sequence of ScFv 1;
b) a heavy chain variable region comprising HCDR3, HCDR1, and/or HCDR2 sequence of ScFv 2;
c) a heavy chain variable region comprising HCDR3, HCDR1, and/or HCDR2 sequence of ScFv 3,
d) a heavy chain variable region comprising HCDR3, HCDR1, and/or HCDR2 sequence of ScFv 4, and
e) a heavy chain variable region comprising HCDR3, HCDR1, and/or HCDR2 sequence of ScFv 5,
f) a heavy chain variable region comprising HCDR3, HCDR1, and/or HCDR2 sequence of ScFv 6, and
g) a heavy chain variable region comprising HCDR3, HCDR1, and/or
HCDR2 sequence of ScFv 7.

4. The antibody or antigen-binding fragment thereof of claim 2, comprising one or more members selected from the group consisting of:
a) a light chain variable region comprising LCDR1, LCDR2, and/or LCDR3 sequence of ScFv 1,
b) a light chain variable region comprising LCDR1, LCDR2, and/or LCDR3 sequence of ScFv 2,
c) a light chain variable region comprising LCDR1, LCDR2, and/or LCDR3 sequence of ScFv 3,
d) a light chain variable region comprising LCDR1, LCDR2, and/or LCDR3 sequence of ScFv 4,
e) a light chain variable region comprising LCDR1, LCDR2, and/or LCDR3 sequence of ScFv 5,
f) a light chain variable region comprising LCDR1, LCDR2, and/or LCDR3 sequence of ScFv 6, and
g) a light chain variable region comprising LCDR1, LCDR2, and/or LCDR3 sequence of ScFv 7.

5. An antibody or antigen-binding fragment thereof comprising:
a) any of the heavy chain variable region of claim 3, and
b) any of the light chain variable region of claim 4.

6. The antibody or antigen-binding fragment thereof of claim 2, comprising a heavy chain variable region comprising:
a) a heavy chain CDR1 selected from the group consisting of ScFv 1 HCDR1, ScFv 2 HCDR1, ScFv 3 HCDR1, ScFv 4 HCDR1, ScFv 5 HCDR1, ScFv 6 HCDR1, ScFv 7 HCDR1;
b) a heavy chain CDR2 selected from the group consisting of ScFv 1 HCDR2, ScFv 2 HCDR2, ScFv 3 HCDR2, ScFv 4 HCDR2, ScFv 5 HCDR2, ScFv 6 HCDR2, ScFv 7 HCDR2; and
c) a heavy chain CDR3 selected from the group consisting of ScFv 1 HCDR3, ScFv 2 HCDR3, ScFv 3 HCDR3, ScFv 4 HCDR3, ScFv 5 HCDR3, ScFv 6 HCDR3, ScFv 7 HCDR3.

7. The antibody or antigen-binding fragment thereof of claim 2, comprising a light chain variable region comprising:
a) a light chain CDR1 selected from the group consisting of ScFv 1 LCDR1, ScFv 2 LCDR1, ScFv 3 LCDR1, ScFv 4 LCDR1, ScFv 5 LCDR1, ScFv 6 LCDR1, ScFv 7 LCDR1,
b) a light chain CDR2 selected from the group consisting of ScFv 1 LCDR2, ScFv 2 LCDR2, ScFv 3 LCDR2, ScFv 4 LCDR2, ScFv 5 LCDR2, ScFv 6 LCDR2, ScFv 7 LCDR2, and
c) a light chain CDR3 selected from the group consisting of ScFv 1 LCDR3, ScFv 2 LCDR3, ScFv 3 LCDR3, ScFv 4 LCDR3, ScFv 5 LCDR3, ScFv 6 LCDR3, and ScFv 7 LCDR3.

8. An antibody or antigen-binding fragment thereof comprising:
a) the heavy chain variable region of claim 6, and
b) the light chain variable region of claim 7.

9. An antibody or antigen-binding fragment thereof comprising an amino acid sequence selected from the group consisting of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 51, SEQ ID NO: 52, SEQ ID NO: 53, SEQ ID NO: 54, SEQ ID NO: 55, and SEQ ID NO: 58.

10. The antibody or antigen-binding fragment thereof of claim 9, comprising a heavy chain variable region selected from the group consisting of:
a) a heavy chain variable region comprising SEQ ID NO: 20, SEQ ID NO: 21, and/or SEQ ID NO: 22;
b) a heavy chain variable region comprising SEQ ID NO: 26, SEQ ID NO: 27, and/or SEQ ID NO: 28;
c) a heavy chain variable region comprising SEQ ID NO: 32, SEQ ID NO: 33, and/or SEQ ID NO: 34;
d) a heavy chain variable region comprising SEQ ID NO: 38, SEQ ID NO: 39, and/or SEQ ID NO: 40,
e) a heavy chain variable region comprising SEQ ID NO: 44, SEQ ID NO: 45, and/or SEQ ID NO: 46,
f) a heavy chain variable region comprising SEQ ID NO: 50, SEQ ID NO: 51, and/or SEQ ID NO: 52, and
g) a heavy chain variable region comprising SEQ ID NO: 56, SEQ ID NO: 57, and/or SEQ ID NO: 58.

11. The antibody or antigen-binding fragment thereof according to claim 10 wherein the heavy chain variable region is selected from the group consisting of:
a) the heavy chain variable region comprising SEQ ID NO: 20, SEQ ID NO: 21, and SEQ ID NO: 22;
b) the heavy chain variable region comprising SEQ ID NO: 26, SEQ ID NO: 27, and SEQ ID NO: 28;
c) the heavy chain variable region comprising SEQ ID NO: 32, SEQ ID NO: 33, and SEQ ID NO: 34;
d) the heavy chain variable region comprising SEQ ID NO: 38, SEQ ID NO: 39, and SEQ ID NO: 40,
e) the heavy chain variable region comprising SEQ ID NO: 44, SEQ ID NO: 45, and SEQ ID NO: 46,
f) the heavy chain variable region comprising SEQ ID NO: 50, SEQ ID NO: 51, and SEQ ID NO: 52, and
g) the heavy chain variable region comprising SEQ ID NO: 56, SEQ ID NO: 57, and SEQ ID NO: 58.

12. The antibody or antigen-binding fragment thereof according to claim 11 wherein the heavy chain variable region is selected from the group consisting of
a) the heavy chain variable region comprising SEQ ID NO: 60;
b) the heavy chain variable region comprising SEQ ID NO: 62;
c) the heavy chain variable region comprising SEQ ID NO:64;
d) the heavy chain variable region comprising SEQ ID NO: 66;
e) the heavy chain variable region comprising SEQ ID NO: 68;
f) the heavy chain variable region comprising SEQ ID NO: 70, and
g) the heavy chain variable region comprising SEQ ID NO:72.

13. The antibody or antigen-binding fragment thereof according to claim 10, further comprising a light chain variable region is selected from the group consisting of:
a) a light chain variable region of comprising SEQ ID NO: 17, SEQ ID NO: 18, and/or SEQ ID NO: 19,
b) a light chain variable region comprising SEQ ID NO: 23, SEQ ID NO: 24, and/or SEQ ID NO: 25,
c) a light chain variable region comprising SEQ ID NO: 29, SEQ ID NO: 30, and/or SEQ ID NO: 31,
d) a light chain variable region comprising SEQ ID NO: 35, SEQ ID NO: 36, and/or SEQ ID NO: 37,
e) a light chain variable region comprising SEQ ID NO: 41, SEQ ID NO: 42, and/or SEQ ID NO: 43,
f) a light chain variable region comprising SEQ ID NO: 47, SEQ ID NO: 48, and/or SEQ ID NO: 49, and
g) a light chain variable region comprising SEQ ID NO: 53, SEQ ID NO: 54, and/or SEQ ID NO: 55.

14. The antibody or antigen-binding fragment thereof according to claim 13 wherein the light chain variable region is selected from the group consisting of:
a) the light chain variable region comprising SEQ ID NO: 17, SEQ ID NO: 18, and SEQ ID NO: 19,
b) the light chain variable region comprising SEQ ID NO: 23, SEQ ID NO: 24, and SEQ ID NO: 25,
c) the light chain variable region comprising SEQ ID NO: 29, SEQ ID NO: 30, and SEQ ID NO: 31,
d) the light chain variable region comprising SEQ ID NO: 35, SEQ ID NO: 36, and SEQ ID NO: 37,
e) the light chain variable region comprising SEQ ID NO: 41, SEQ ID NO: 42, and SEQ ID NO: 43,
f) the light chain variable region comprising SEQ ID NO: 47, SEQ ID NO: 48, and SEQ ID NO: 49, and
g) the light chain variable region comprising SEQ ID NO: 53, SEQ ID NO: 54, and SEQ ID NO: 55.

15. The antibody or antigen-binding fragment thereof according to claim 14, wherein the light chain variable region comprises SEQ ID NO: 17, SEQ ID NO: 18, and SEQ ID NO: 19; and the heavy chain variable region comprises SEQ ID NO: 20, SEQ ID NO: 21, and SEQ ID NO: 22.

16. The antibody or antigen-binding fragment thereof according to claim 14, wherein the light chain variable region comprises SEQ ID NO: 23, SEQ ID NO: 24, and SEQ ID NO: 25; and the heavy chain variable region comprises SEQ ID NO: 26, SEQ ID NO: 27, and SEQ ID NO: 28.

17. The antibody or antigen-binding fragment thereof according to claim 14, wherein the light chain variable region comprises SEQ ID NO: 29, SEQ ID NO: 30, and SEQ ID NO: 31; and the heavy chain variable region comprises SEQ ID NO: 32, SEQ ID NO: 33, and SEQ ID NO: 34.

18. The antibody or antigen-binding fragment thereof according to claim 14, wherein the light chain variable region comprises SEQ ID NO: 35, SEQ ID NO: 36, and SEQ ID NO: 37; and the heavy chain variable region comprises SEQ ID NO: 38, SEQ ID NO: 39, and SEQ ID NO: 40.

19. The antibody or antigen-binding fragment thereof according to claim 14, wherein the light chain variable region comprises SEQ ID NO: 41, SEQ ID NO: 42, and SEQ ID NO: 43; and the heavy chain variable region comprises SEQ ID NO: 44, SEQ ID NO: 45, and SEQ ID NO: 46.

20. The antibody or antigen-binding fragment thereof according to claim 14, wherein the light chain variable region comprises SEQ ID NO: 47, SEQ ID NO: 48, and SEQ ID NO: 49; and the heavy chain variable region comprises SEQ ID NO: 50, SEQ ID NO: 51, and SEQ ID NO: 52.

21. The antibody or antigen-binding fragment thereof according to claim 14, wherein the light chain variable region comprises SEQ ID NO: 53, SEQ ID NO: 54, and SEQ ID NO: 55; and the heavy chain variable region comprises SEQ ID NO: 56, SEQ ID NO: 57, and SEQ ID NO: 58.

22. The antibody or antigen-binding fragment thereof according to claim 14 wherein the light chain variable region is selected from the group consisting of:
a) the light chain variable region comprising SEQ ID NO:59,
b) the light chain variable region comprising SEQ ID NO: 61,
c) the light chain variable region comprising SEQ ID NO:63,
d) the light chain variable region comprising SEQ ID NO: 65,
e) the light chain variable region comprising SEQ ID NO: 67,
f) the light chain variable region comprising SEQ ID NO: 69, and
g) the light chain variable region comprising SEQ ID NO: 71.

23. (canceled)

24. (canceled)

25. (canceled)

26. (canceled)

27. (canceled)

28. (canceled)

29. (canceled)

30. The antibody or antigen-binding fragment thereof of claim 9, comprising a heavy chain variable region comprising:
a) a heavy chain CDR1 selected from the group consisting of SEQ ID NO: 20, SEQ ID NO: 26, SEQ ID NO: 32, SEQ ID NO: 38, and SEQ ID NO: 44:
b) a heavy chain CDR2 selected from the group consisting of SEQ ID NO: 21, SEQ ID NO: 27, SEQ ID NO: 33, SEQ ID NO: 39, and SEQ ID NO: 45; and
c) a heavy chain CDR3 selected from the group consisting of SEQ ID NO: 22, SEQ ID NO: 28, SEQ ID NO: 34, SEQ ID NO: 40, SEQ ID NO: 46, SEQ ID NO: 52, and SEQ ID NO: 58.

31. The antibody or antigen-binding fragment thereof according to claim 30 wherein the heavy chain variable region is selected from the group consisting of:
a) the heavy chain variable region comprising SEQ ID NO: 20, SEQ ID NO: 21, and SEQ ID NO: 22;
b) the heavy chain variable region comprising SEQ ID NO: 26, SEQ ID NO: 27, and SEQ ID NO: 28;
c) the heavy chain variable region comprising SEQ ID NO: 32, SEQ ID NO: 33, and SEQ ID NO: 34;
d) the heavy chain variable region comprising SEQ ID NO: 38, SEQ ID NO: 39, and SEQ ID NO: 40,
e) the heavy chain variable region comprising SEQ ID NO: 44, SEQ ID NO: 45, and SEQ ID NO: 46,
f) the heavy chain variable region comprising SEQ ID NO: 50, SEQ ID NO: 51, and SEQ ID NO: 52, and
g) the heavy chain variable region comprising SEQ ID NO: 56, SEQ ID NO: 57, and SEQ ID NO: 58.

32. The antibody or antigen-binding fragment thereof according to claim 31 wherein the heavy chain variable region is selected from the group consisting of
a) the heavy chain variable region comprising SEQ ID NO: 60;
b) the heavy chain variable region comprising SEQ ID NO: 62;
c) the heavy chain variable region comprising SEQ ID NO:64;
d) the heavy chain variable region comprising SEQ ID NO: 66;
e) the heavy chain variable region comprising SEQ ID NO: 68;
f) the heavy chain variable region comprising SEQ ID NO: 70, and
g) the heavy chain variable region comprising SEQ ID NO:72.

33. The antibody or antigen-binding fragment thereof according to claim 30 further comprising a light chain variable region comprising:
a) a light chain CDR1 selected from the group consisting of SEQ ID NO: 17, SEQ ID NO: 23, SEQ ID NO: 35, SEQ ID NO: 47, and SEQ ID NO: 53;
b) a light chain CDR2 selected from the group consisting of SEQ ID NO: 18, SEQ ID NO: 24, SEQ ID NO: 36, SEQ ID NO: 48, and SEQ ID NO: 54; and
c) a light chain CDR3 selected from the group consisting of SEQ ID NO: 19, SEQ ID NO: 25, SEQ ID NO: 37, SEQ ID NO: 43, SEQ ID NO: 49, and SEQ ID NO: 55.

34. The antibody or antigen-binding fragment thereof according to claim 33 wherein the heavy chain variable region is selected from the group consisting of
a) the light chain variable region comprising SEQ ID NO: 17, SEQ ID NO: 18, and SEQ ID NO: 19,
b) the light chain variable region comprising SEQ ID NO: 23, SEQ ID NO: 24, and SEQ ID NO: 25,
c) the light chain variable region comprising SEQ ID NO: 29, SEQ ID NO: 30, and SEQ ID NO: 31,
d) the light chain variable region comprising SEQ ID NO: 35, SEQ ID NO: 36, and SEQ ID NO: 37,
e) the light chain variable region comprising SEQ ID NO: 41, SEQ ID NO: 42, and SEQ ID NO: 43,
f) the light chain variable region comprising SEQ ID NO: 47, SEQ ID NO: 48, and SEQ ID NO: 49, and
g) the light chain variable region comprising SEQ ID NO: 53, SEQ ID NO: 54, and SEQ ID NO: 55.

35. The antibody or antigen-binding fragment thereof according to claim 34 wherein the light chain variable region is selected from the group consisting of:
a) the light chain variable region comprising SEQ ID NO:59,
b) the light chain variable region comprising SEQ ID NO: 61,
c) the light chain variable region comprising SEQ ID NO:63,
d) the light chain variable region comprising SEQ ID NO: 65,
e) the light chain variable region comprising SEQ ID NO: 67,
f) the light chain variable region comprising SEQ ID NO: 69, and
g) the light chain variable region comprising SEQ ID NO: 71.

36. The antibody or antigen-binding fragment thereof as recited in claim 1, which is a monoclonal antibody, a polyclonal antibody, a bispecific antibody, a chimeric antibody, a humanized antibody, a recombinant antibody, a human antibody, a labeled antibody, a bivalent antibody, or an anti-idiotypic antibody.

37. The antibody or antigen-binding fragment thereof as recited in claim 1 which is a camelized single domain antibody, a diabody, a scFv, an scFv dimer, a BsFv, a dsFv, a (dsFv)₂, a dsFv-dsFv′, an Fv fragment, a Fab, a Fab′, a F(ab′)₂, a ds diabody, a nanobody, a domain antibody, or a bivalent domain antibody.

38. The antibody or antigen-binding fragment thereof as recited in claim 1, further comprising an immunoglobulin constant region.

39. (canceled)

40. A complex comprising an antibody or antigen-binding fragment thereof as recited in claim 1 bound to hER-α36 as set forth in SEQ ID NO:1 or a fragment of hER-α36.

41. An isolated polypeptide comprising one or more amino acid sequences of the antibody or antigen-binding fragment there of as recited in claim 1.

42. An isolated polynucleotide encoding the polypeptide of claim 41.

43. An isolated vector comprising the polynucleotide of claim 42.

44. An isolated host cell comprising the vector of claim 43.

45. A method of expressing a polypeptide comprising one or more amino acid sequences of claim 41 comprising culturing the isolated host cell of claim 44 under conditions in which the polynucleotide of claim 42 is expressed.

46. A pharmaceutical composition comprising the antibody or antigen-binding fragment thereof as recited in claim 1 and one or more pharmaceutically acceptable carriers.

47. (canceled)

48. A pharmaceutical composition comprising the antibody or antigen-binding fragment thereof as recited in claim 1 linked to or in combination with one or more chemotherapeutic agents.

49. (canceled)

50. A pharmaceutical composition comprising the antibody or antigen-binding fragment thereof as recited in claim 1 in combination with one or more therapeutic agents.

51. (canceled)

52. (canceled)

53. A composition comprising the antibody or antigen-binding fragment thereof as recited in claim 1 linked to one or more conjugates.

54. (canceled)

55. A method of modulating the ER-α36 activities in a cell comprising exposing a cell expressing ER-α36 to the antibodies and antigen-binding fragment as recited in claim 1.

56. A method of detecting the presence of ER-α36 in a sample comprising exposing a sample to the antibodies and antigen-binding fragment as recited in claim 1 and determining the presence of ER-α36.

57. A method of treating or preventing a condition associated with ER-α36 in a subject comprising administering the subject a therapeutic effective amount of a pharmaceutical composition comprising the antibody or antigen-binding fragment thereof as recited in claim 1.

58. (canceled)

59. The method of claim 57 wherein the condition includes bone loss, bone fracture, osteoporosis, menopause, premenstrual syndrome, endometriosis, uterine disease, impotence, sexual dysfunctions, increased levels of LDL cholesterol, cardiovascular diseases, atherosclerosis, vascular smooth muscle cell proliferation, depression resulting from an estrogen deficiency, perimenopausal depression, post-partum depression, immune deficiency, auto immune diseases, inflammation, inflammatory condition, asthma and cancerous condition.

60. (canceled)

61. (canceled)

62. The method of claim 59 wherein the cancerous condition is selected from the group consisting of squamous cell cancer, lung cancer (e.g., small cell lung cancer, non-small cell lung cancer (NSCLC), adenocarcinoma of the lung, or squamous cell carcinoma of the lung), cancer of the peritoneum, liver cancer (e.g., hepatocellular carcinoma/hepatoma), gastric or stomach cancer (e.g., gastrointestinal cancer), pancreatic cancer, brain tumor (e.g., glioblastoma/glioblastoma multiforme (GBM), non-glioblastoma brain tumor, or meningioma), glioma (e.g., ependymoma, astrocytoma, anaplastic astrocytoma, oligodendroglioma, or mixed glioma such as oligoastrocytoma), cervical cancer, ovarian cancer, liver cancer (e.g., hepatoblastoma, hepatocellular carcinoma/hepatoma, or hepatic carcinoma), bladder cancer (e.g., urothelial cancer), breast cancer, colon cancer, colorectal cancer, rectal cancer, endometrial or uterine carcinoma, salivary gland carcinoma, kidney or renal cancer (e.g., rhabdoid tumor of the kidney), prostate cancer, vulval cancer, penile cancer, anal cancer (e.g., anal squamous cell carcinoma), thyroid cancer, head and neck cancer (e.g., nasopharyngeal cancer), skin cancer (e.g., melanoma or squamous cell carcinoma), osteosarcoma, Ewing's sarcoma, chondrosarcoma, soft tissue sarcoma (e.g., rhabdomyosarcoma, fibrosarcoma, Kaposi's sarcoma), carcinoid cancer, eye cancer (e.g., retinoblastoma), mesothelioma, lymphocytic/lymphoblastic leukemia (e.g., acute lymphocytic/lymphoblastic leukemia (ALL) of both T-cell lineage and B-cell precursor lineage, chronic lymphoblastic/lymphocytic leukemia (CLL), acute myelogenous/myeloblastic leukemia (AML), including mast cell leukemia, chronic myelogenous/myelocytic/myeloblastic leukemia (CML), hairy cell leukemia (HCL), Hodgkin's disease, non-Hodgkin's lymphoma, chronic myelomonocytic leukemia (CMML), follicular lymphoma (FL), diffuse large B cell lymphoma (DLCL), mantle cell lymphoma (MCL), Burkitt's lymphoma (BL), mycosis fungoides, Sezary syndrome, cutaneous T-cell lymphoma, mast cell neoplasm, medulloblastoma, nephroblastoma, solitary plasmacytoma, myelodysplastic syndrome, chronic and non-chronic myeloproliferative disorder, central nervous system tumor, pituitary adenoma, vestibular schwannoma, primitive neuroectodermal tumor, ependymoma, choroid plexus papilloma, polycythemia vera, thrombocythemia, idiopathic myelofibrosis, and pediatric cancers such as pediatric sarcomas (e.g., neuroblastoma, rhabdomyosarcoma, and osteosarcoma).

63. The method of claim 57 wherein the pharmaceutical composition is administered through a parenteral route which is subcutaneous, intraperitoneal, intravenous, intramuscular, or intradermal injection; or a non-parenteral route which is transdermal, oral, intranasal, intraocular, sublingual, rectal, or topical.

64. The method of claim 57 wherein the therapeutic effective amount is about 0.01 mg/kg to about 100 mg/kg.

65. The method of claim 64 wherein the therapeutic effective amount selected from the group consisting of about 0.01 mg/kg, about 0.5 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, and about 100 mg/kg.

66. A method of determine the status of a condition associated ER-α36 in a subject comprising measuring the level of ER-α36 using the antibody or antigen-binding fragment thereof as recited in claim 1.
